(19) United States
(12) Patent Application Publication
(10) Pub. No.: US 2025/0161233 A1
(43) Pub. Date: May 22, 2025

(54) VINYLATION COUPLED DERIVATIVE OF BETA-ELEMENE, AND PREPARATION METHOD AND USE THEREOF IN PREPARATION OF ANTITUMOR DRUG

(71) Applicant: HANGZHOU NORMAL UNIVERSITY, Hangzhou, Zhejiang (CN)

(72) Inventors: Tian XIE, Hangzhou, Zhejiang (CN); Yang YE, Hangzhou, Zhejiang (CN); Bing XU, Hangzhou, Zhejiang (CN); Xiangyang YE, Hangzhou, Zhejiang (CN)

(73) Assignee: HANGZHOU NORMAL UNIVERSITY, Hangzhou, Zhejiang (CN)

(21) Appl. No.: 18/476,988

(22) PCT Filed: Nov. 17, 2022
(86) PCT No.: PCT/CN2022/132420

§ 371 Date: Jan. 31, 2025

(30) Foreign Application Priority Data
Feb. 28, 2022 (CN) 202210185780.6

(51) Int. Cl. A61K 31/085; A61K 31/235; A61K 31/277; A61K 31/404; A61K 31/4402; A61P 35/00; C07C 41/30; C07C 291/00; C07D 213/643

(52) U.S. Cl. CPC A61K 31/085; A61K 31/235; A61K 31/277; A61K 31/404; A61K 31/4402; A61P 35/00; C07C 41/30; C07C 291/00; C07D 213/643

(57) ABSTRACT

The present disclosure provides a vinylation coupled derivative of β-elemene, and a preparation method and use thereof in preparation of an antitumor drug. The present disclosure provides a vinylation coupled derivative of β-elemene having a structure shown in Formula (I), a pharmaceutical composition and a hydrate including a compound shown in Formula (I), as well as an isotopic derivative, a chiral isomer, a variant, a salt, a prodrug, and a preparation of the compound shown in Formula (I). The present disclosure further provides a preparation method and use of the vinylation coupled derivative of β-elemene, and an inhibitory activity of the derivative on proliferation of various tumor cell lines. The vinylation coupled derivative of β-elemene is expected to be an antitumor drug candidate for treating colon cancer and lung cancer.

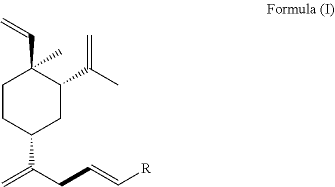

### CROSS REFERENCE TO RELATED APPLICATION

The present disclosure claims priority to the Chinese Patent Application CN202210185780.6 filed to the China National Intellectual Property Administration (CNIPA) on Feb. 28, 2022 and entitled “VINYLATION COUPLED DERIVATIVE OF BETA-ELEMENE, AND PREPARATION METHOD AND USE THEREOF IN PREPARATION OF ANTITUMOR DRUG”, which is incorporated herein by reference in its entirety.

### TECHNICAL FIELD

The present disclosure relates to the technical field of preparation of β-elemene derivatives, in particular to a vinylation coupled derivative of β-elemene, and a preparation method and use thereof in preparation of an antitumor drug.

### BACKGROUND

Elemene as a type of small-molecule volatile oil compound is mainly extracted from the tuber of Curcuma wenyujin. Elemene reported in existing literatures mainly includes α-elemene, (±)-β-elemene, γ-elemene, and δ-elemene. The (−)-β-elemene is the main active ingredient exerting anti-tumor effects, has a broad-spectrum anti-tumor activity, and shows certain curative effects on various cancers, such as liver cancer, breast cancer, and lung cancer. At present, a variety of preparations with the elemene as a main component have achieved certain anticancer effects in clinical practice.

β-Elemene is one of the few new anti-tumor drugs with independent intellectual property rights in China. The research on the derivatives of β-elemene has made great progress in recent years. According to the research results of a structure-activity relationship, the double bond and the backbone of β-elemene have an important influence on its activity. The activities of β-elemene compounds without three double bonds being destroyed are not greatly affected. However, low polarity, poor water solubility, and low oral bioavailability limit the clinical applications of elemene. Therefore, it is necessary to modify the structure of elemene to obtain elemene derivatives with desirable water solubility, higher oral bioavailability, better biological activity than that of elemene, and less toxic and side effects.

The structural modification of β-elemene has been developed since the 1970s. There are more than 150 kinds of derivatives of β-elemene that have been synthesized, involving several major categories such as reduction and oxidation, sulfur-containing, nitrogen-containing, esters, ethers, alcohols, and glycosides. Currently, there are very few studies on the vinylation coupling of β-elemene.

### SUMMARY

A first purpose of the present disclosure is to address the deficiencies of the prior art, and to construct a series of vinylation coupled derivatives of β-elemene with β-elemene as a matrix. The present disclosure provides a vinylation coupled derivative of β-elemene, and researches its antitumor activity, so as to develop a new antitumor drug.

The present disclosure provides a vinylation coupled derivative of β-elemene, or an optical isomer, a racemate, a single enantiomer, and a possible diastereomer thereof, or a pharmaceutically acceptable salt, a prodrug, a deuterated derivative, a hydrate, and a solvate thereof, where the vinylation coupled derivative of β-elemene has a structure shown in formula (I):

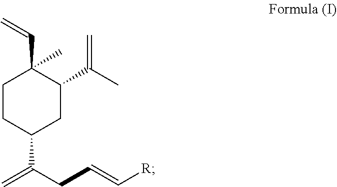

in Formula (I):
R is any one independently selected from the group consisting of the following structural fragments:

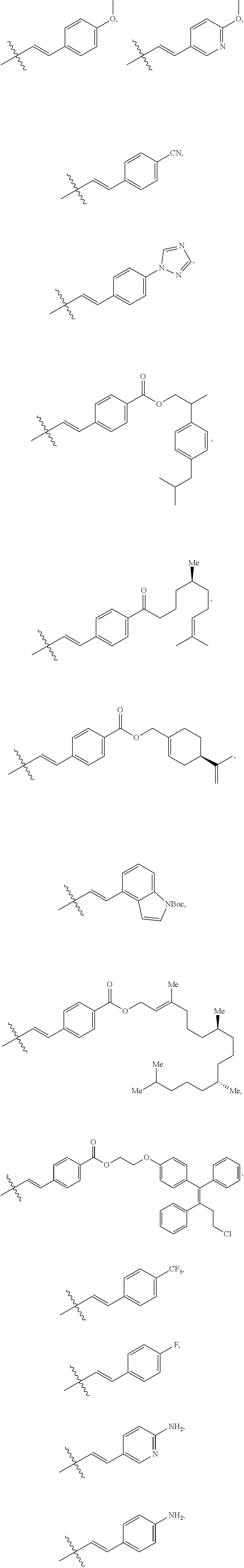

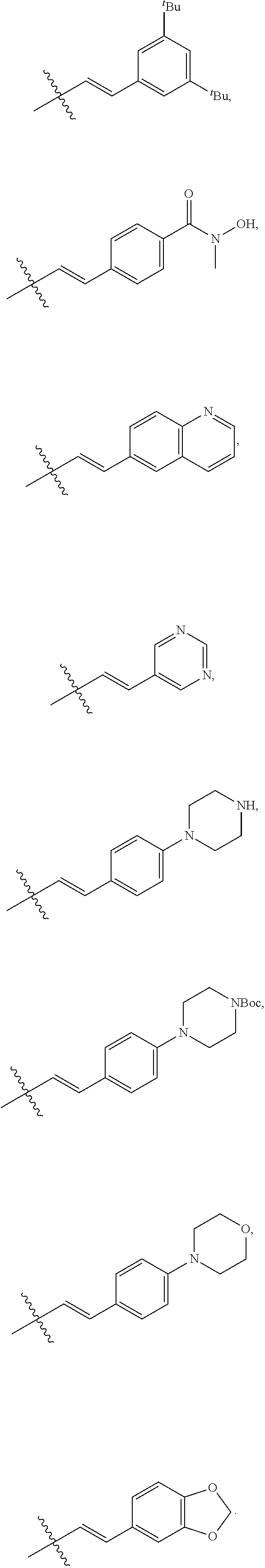

Further, the vinylation coupled derivative of β-elemene has a structure shown in any one of the Compounds 1 to 22:

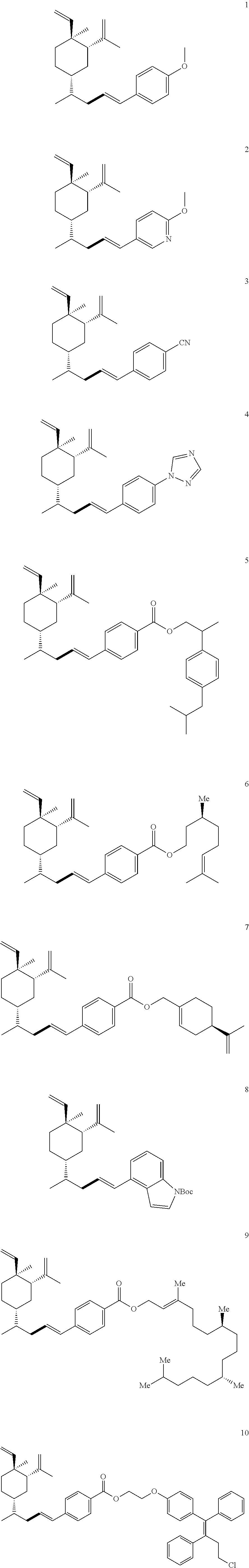

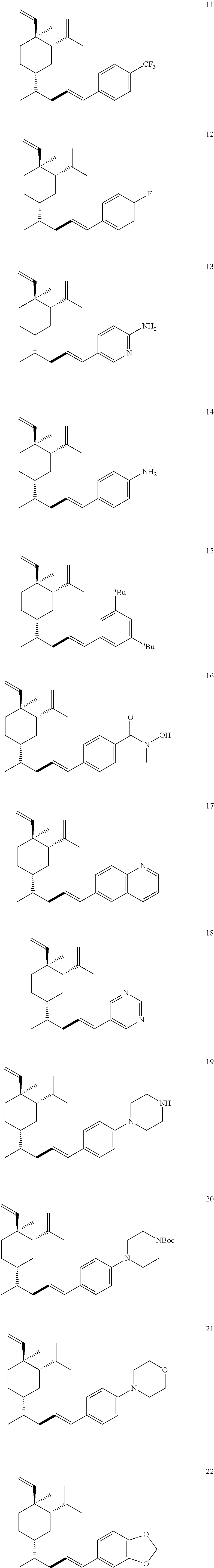

A second purpose of the present disclosure is to provide a preparation method of the vinylation coupled derivative of β-elemene.

In the present disclosure, the following synthetic route can be adopted for the vinylation coupled derivative of β-elemene having the structure shown in Formula (I):

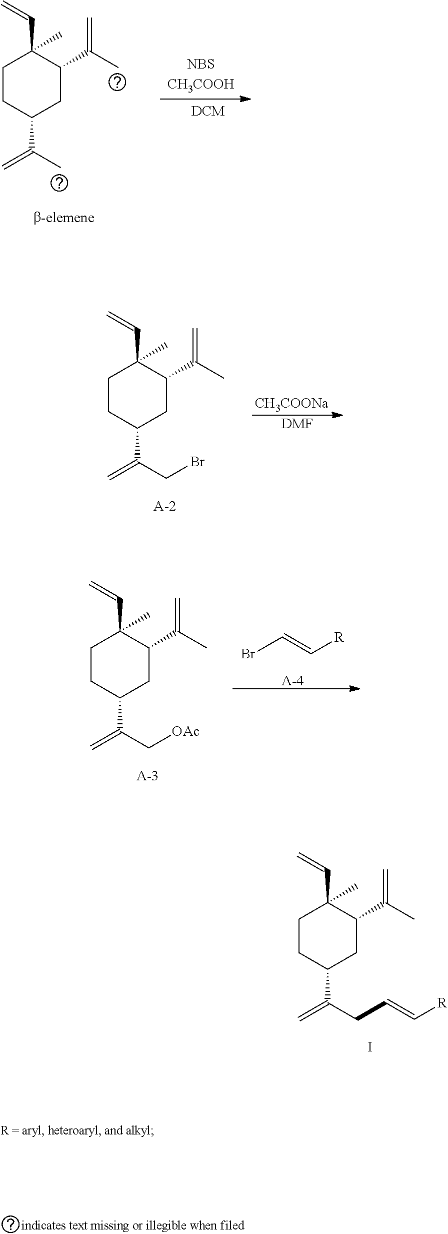

and
the preparation method includes the following steps:
(1) subjecting β-elemene A-1 to bromine substitution at an allyl position 13 to obtain an intermediate A-2;
(2) subjecting the intermediate A-2 to selective nucleophilic substitution to obtain an intermediate A-3; and
(3) allowing the intermediate A-3 and alkenyl bromide A-4 to have vinylation coupling to obtain the vinylation coupled derivative of β-elemene having a structure shown in Formula (I).

In the present disclosure, the vinylation coupled derivative of β-elemene having a structure shown in Formula (I) is prepared by the above method. However, the conditions of the above methods, such as reactants, solvents, amounts of compounds used, reaction temperature, time required for reaction, are not limited to the above explanations. The compound can also be conveniently prepared by optionally combining various synthetic methods described in this description or known in the art. Such combinations can be easily conducted by those skilled in the art to which the present disclosure pertains.

In the present disclosure, the step (1) of each synthetic route can adopt the prior art, as disclosed in the patent CN110683932A.

A third purpose of the present disclosure is to provide use of the vinylation coupled derivative of β-elemene, or an optical isomer, a racemate, a single enantiomer, and a possible diastereomer thereof, or a pharmaceutically acceptable salt, a prodrug, a deuterated derivative, a hydrate, and a solvate thereof in preparation of an antitumor drug.

A fourth purpose of the present disclosure is to provide an antitumor drug a safe and effective dosage of the vinylation coupled derivative of β-elemene, or an optical isomer, a racemate, a single enantiomer, and a possible diastereomer thereof, or a pharmaceutically acceptable salt, a prodrug, a deuterated derivative, a hydrate, and a solvate thereof.

Preferably, the antitumor drug may further include a pharmaceutically acceptable carrier.

Preferably, in the use and the antitumor drug, the tumor includes colon cancer and lung cancer.

In the present disclosure, the compound has the activity of inhibiting the proliferation of various tumor cell lines. Therefore, the compound and its various crystal forms, pharmaceutically acceptable inorganic or organic salts, hydrates or solvates, and pharmaceutical compositions including the compound of the present disclosure as a main active ingredient can be used for the treatment, prevention, and alleviation of various diseases, including various cancers.

In the present disclosure, the pharmaceutical composition includes the compound of the present disclosure or a pharmaceutically acceptable salt thereof and a pharmaceutically acceptable excipient or carrier within a safe and effective amount range. Herein, the “safe and effective dose” refers to: an amount of the compound is sufficient to improve the condition obviously without causing serious side effects. Generally, the pharmaceutical composition includes 1 mg to 2,000 mg of the compound of the present disclosure/dose, more preferably 5 mg to 1,000 mg of the compound of the present disclosure/dose. Preferably, the “one dose” refers to one capsule or one tablet.

The “pharmaceutically acceptable carrier” refers to: one or more compatible solid or liquid fillers or gel substances, which are suitable for human and must have sufficient purity and low enough toxicity. The “compatibility” here means that each component in the composition can be blended with the compound of the present disclosure and with each other without significantly reducing the efficacy of the compound. Some examples of the pharmaceutically acceptable carrier include: cellulose and derivatives thereof (such as sodium carboxymethylcellulose, sodium ethylcellulose, and cellulose acetate), gelatin, talc, solid lubricants (such as stearic acid and magnesium stearate), calcium sulfate, vegetable oils (such as soybean oil, sesame oil, peanut oil, and olive oil), polyols (such as propylene glycol, glycerin, mannitol, and sorbitol), emulsifiers, wetting agents (such as ten sodium lauryl sulfate), coloring agents, flavoring agents, stabilizers, antioxidants, preservatives, and pyrogen-free water.

In the present disclosure, there is no special limitation on an administration mode of the compound or the pharmaceutical composition, and representative administration modes include (but not limited to): oral administration, intratumoral administration, rectal administration, parenteral administration (intravenous, intramuscular, or subcutaneous administration), and topical administration.

A solid dosage form for the oral administration includes a capsule, a tablet, a pill, a powder, and a granule. In the solid dosage form, the active compound is mixed with at least one conventional inert excipient (or carrier), such as sodium citrate or dicalcium phosphate, or with the following ingredients: (a) fillers or solubilizers, such as starch, lactose, sucrose, glucose, mannitol, and silicic acid; (b) binders, such as hydroxymethylcellulose, alginate, gelatin, polyvinylpyrrolidone, sucrose, and arabic gum; (c) humectants, such as glycerin; (d) disintegrants, such as agar, calcium carbonate, potato or tapioca starch, alginic acid, certain complex silicate, and sodium carbonate; (e) retarding solvent, such as paraffin; (f) absorption accelerators, such as quaternary ammonium compounds; (g) wetting agents, such as cetyl alcohol and glyceryl monostearate; (h) adsorbents, such as kaolin; (i) lubricants, such as talc, calcium stearate, magnesium stearate, solid polyethylene glycol, and sodium lauryl sulfate, or mixtures thereof. In capsules, tablets, and pills, the dosage form may further include a buffer.

Solid dosage forms such as tablets, dragees, capsules, pills, and granules can be prepared with coatings and shell materials such as enteric coatings and others well known in the art. These dosage forms may contain an opacifying agent, and the active compound or release of the compound from such compositions may be in a delayed manner at a site within the alimentary canal. Examples of usable embedding components are polymeric substances and waxy substances. The active compound can also be in a microencapsulated form, if desired, with one or more of the above-mentioned excipients.

Liquid dosage forms for oral administration include pharmaceutically acceptable emulsions, solutions, suspensions, syrups, or tinctures. Liquid dosage forms may include, in addition to the active compound, inert diluents such as water or other solvents, solubilizers and emulsifiers conventionally used in the art. For example, ethanol, isopropanol, ethyl carbonate, ethyl acetate, propylene glycol, 1,3-butanediol, dimethylformamide, and oils, especially cottonseed oil, peanut oil, corn germ oil, olive oil, castor oil, and sesame oil or a mixture of these substances can be used.

Besides such inert diluents, the composition may also include adjuvants, such as wetting agents, emulsifiers, and suspending, sweetening, flavoring, and perfuming agents.

Suspensions, in addition to the active compounds, may include suspending agents such as ethoxylated isooctadecanol, polyoxyethylene sorbitol, and sorbitan esters, microcrystalline cellulose, aluminum methoxide, and agar, or mixtures of these substances.

Compositions for parenteral injection may include physiologically acceptable sterile aqueous or anhydrous solutions, dispersions, suspensions, or emulsions as well as sterile powders for reconstitution into sterile injectable solutions or dispersions. Suitable aqueous and non-aqueous carriers, diluents, solvents, or excipients include water, ethanol, polyols, and suitable mixtures thereof.

Dosage forms for topical administration of the compound in the present disclosure include ointments, powders, patches, sprays, and inhalants. The active ingredient is mixed under sterile conditions with a physiologically acceptable carrier and any preservatives, buffers, or propellants which may be necessary.

In the present disclosure, the compound can be administered alone or in combination with other pharmaceutically acceptable compounds.

When using a pharmaceutical composition, a safe and effective dosage of the compound of the present disclosure is applied to a mammal (such as a human) in need of treatment. The dosage during administration is a pharmaceutically effective dosage. For a person weighing 60 kg, a daily dosage is generally 1 mg to 5,000 mg, preferably 5 mg to 2,000 mg. Of course, factors such as an administration route and a health status of the patient should also be considered for the specific dosage, and are within the skill of skilled physicians.

Compared with the prior art, the main advantages of the present disclosure include: the present disclosure provides a vinylation coupled derivative of β-elemene having a structure shown in Formula (I), pharmaceutical compositions and hydrates including the vinylation coupled derivative of β-elemene having a structure shown in Formula (I), as well as isotopic derivatives, chiral isomers, variants, different salts, prodrugs, and preparations of the compound. The present disclosure further provides a preparation method and use of the vinylation coupled derivative of β-elemene, and an inhibitory activity of the derivative on proliferation of various tumor cell lines. The C(sp³)—C(sp²) bond-coupled derivatives of the β-elemene are expected to become anti-tumor drug candidates for the treatment of various cancers, such as lung cancer, liver cancer, colorectal cancer, gastric cancer, prostate cancer, ovarian cancer, breast cancer, or glioma.

### DETAILED DESCRIPTION OF THE EMBODIMENTS

The present disclosure will be further described below with reference to specific examples. It should be understood that these embodiments are only intended to describe the present disclosure, rather than to limit the scope of the present disclosure. In the following examples, the experimental methods in which specific conditions are not stated are generally conducted according to conventional conditions or according to the conditions recommended by the manufacturer.

### Example 1: Preparation of Compound 1

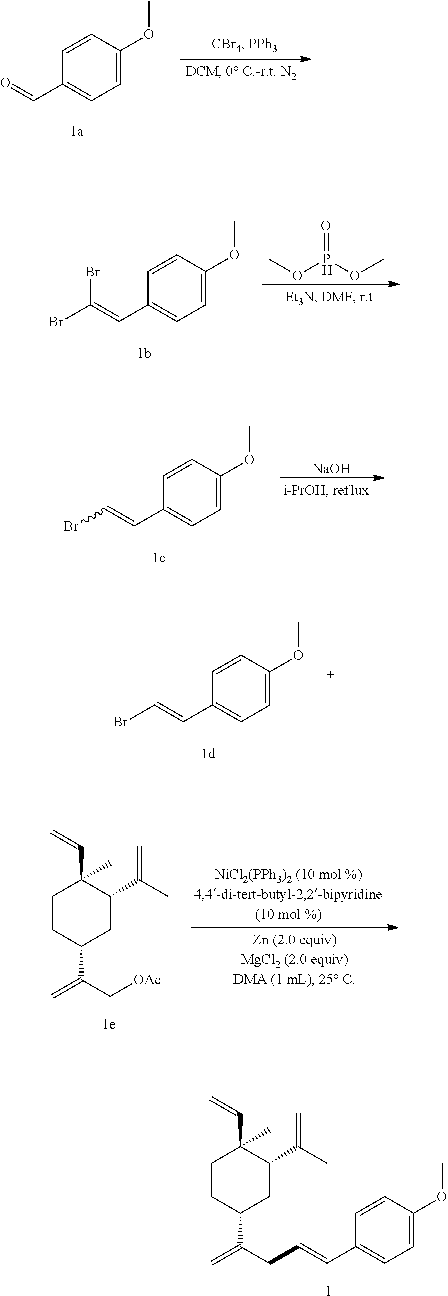

### Intermediate 1d

(1) CBr₄ (30 mmol, 150 mol %) was added to a dichloromethane (DCM, 80 mL) solution of Compound 1a (20 mmol, 100 mol %) at 0° C.; under nitrogen protection, a DCM (70 mL) solution of PPh₃ (60 mmol, 300 mol %) was added dropwise through a constant-pressure dropping funnel into a resulting mixture. A reaction was monitored by TLC. After the reaction was completed, about half of the volume of DCM was removed under reduced pressure, and then petroleum ether (PE, 100 mL) was added to precipitate triphenylphosphine oxide (TPPO). After filtration by diatomaceous earth and evaporation, an obtained residue was dissolved in PE (50 mL) to further precipitate the TPPO. The above steps were repeated several times to obtain a crude dibromide 1b (−20 mmol, 100 mol %), which was directly used in the next step.

(2) An anhydrous N,N-dimethylformamide (DMF, 20 mL) solution of the crude dibromide 1b (−20 mmol, 100 mol %) and NEt₃ (60 mmol, 300 mol %) was added with dimethyl phosphite (60 mmol, 300 mol %) and stirred overnight under room temperature. DMF was distilled off under reduced pressure, an aqueous solution (60 mL) was added, a resulting product was extracted with PE (2×100 mL), an obtained combined organic phase was washed with an aqueous hydrochloric acid solution (1 M, 55 mL), and dried over anhydrous sodium sulfate. The above desiccant was removed by filtration, and a resulting filtrate was concentrated under reduced pressure to obtain a crude product 1c, which was directly used in the next step.

(3) The crude product 1c (−20 mmol, 100 mol %) was dissolved in isopropanol (i-PrOH, 30 mL). The solid sodium hydroxide (17 mmol, 85 mol %) was added to conduct reflux by heating for more than 5 h. A resulting reaction mixture was cooled to room temperature, extracted with n-hexane (100 mL), and an obtained combined organic phase was washed successively with an aqueous hydrochloric acid solution (1 M, 75 mL) and an aqueous solution (2×100 mL), and then dried over anhydrous sodium sulfate. The above desiccant was removed by filtration, an obtained filtrate was concentrated under reduced pressure, and an obtained crude product was purified by silica gel column chromatography (eluted with an ethyl acetate/petroleum ether (0.5%) system) to obtain a white solid 1d (2.65 g, yield 62%). ¹H NMR (500 MHz, CDCl₃) δ 7.25-7.21 (m, 2H), 7.04 (d, J=14.0 Hz, 1H), 6.87-6.83 (m, 2H), 6.61 (d, J=13.9 Hz, 1H), 3.81 (s, 3H).

### Compound 1

The intermediate 1d (0.150 mmol, 1.0 equiv), intermediate 1e (0.300 mmol, 2.0 equiv), a catalyst NiCl₂ (PPh₃)₂ (9.8 mg, 0.015 mmol, 10 mol %), a reducing agent Zn powder (19.6 mg, 0.300 mmol, 2.0 equiv), a ligand 4,4′-di-tert-butyl-2,2′-bipyridine (4.0 mg, 0.015 mmol, 10 mol %), and an additive MgCl₂ (28.6 mg, 0.300 mmol, 2.0 equiv) were added sequentially to an oven-dried Schlenk tube equipped with a magnetic stirrer. The Schlenk tube was evacuated three times through a double-row tube to ensure that the reaction system was under a nitrogen atmosphere, and finally an N,N-dimethylacetamide (DMA, 1.0 mL) solution was added with a syringe and stirred at 25° C. for 12 h. After the reaction was completed, an obtained reaction mixture was directly purified by silica gel column chromatography (eluted with an ethyl acetate/petroleum ether (2%) system) without post-treatment to obtain a colorless oily liquid 1 (43.3 mg, yield 86%). ¹H NMR (500 MHz, CDCl₃) δ 7.31 (d, J=8.7 Hz, 2H), 6.85 (d, J=8.7 Hz, 2H), 6.37 (d, J=15.8 Hz, 1H), 6.08 (dt, J=15.7, 7.1 Hz, 1H), 5.83 (dd, J=17.4, 10.9 Hz, 1H), 4.92 (dd, J=8.1, 1.2 Hz, 1H), 4.89 (s, 1H), 4.87 (s, 1H), 4.83 (dd, J=5.0, 1.3 Hz, 2H), 4.61 (s, 1H), 3.81 (s, 3H), 2.95 (d, J=7.0 Hz, 2H), 2.02 (dd, J=11.8, 4.3 Hz, 2H), 1.72 (s, 3H), 1.64-1.59 (m, 2H), 1.54-1.42 (m, 4H), 1.02 (s, 3H). ¹³C NMR (126 MHz, CDCl₃) δ 158.92, 153.40, 150.38, 147.81, 130.81, 130.65, 127.27, 126.63, 114.06, 112.26, 110.01, 108.76, 55.41, 52.90, 44.30, 40.09, 39.98, 38.83, 33.36, 27.33, 24.93, 16.76. HRMS (ESI) m/z ([M−H]⁻) calcd for C₂₄H₃₁O: 335.2380. Found: 335.2380.

### Example 2: Preparation of Compound 2

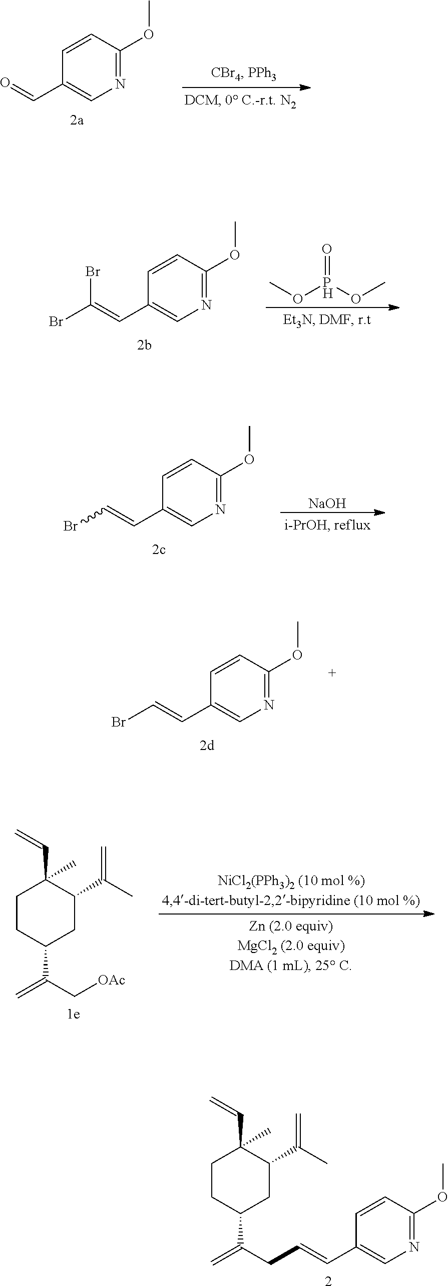

### Intermediate 2d

Referring to the synthesis steps of the intermediate 1d in Example 1, a colorless oily liquid 2d (881 mg, yield 21%) was obtained. ¹H NMR (500 MHz, CDCl₃) δ 8.07 (d, J=2.5 Hz, 1H), 7.55 (dd, J=8.6, 2.5 Hz, 1H), 7.03 (d, J=14.0 Hz, 1H), 6.71 (d, J=8.7 Hz, 1H), 6.66 (d, J=14.0 Hz, 1H), 3.94 (s, 3H).

### Compound 2

Referring to the synthesis steps of the Compound 1 in Example 1, a colorless oily liquid 2 (36.9 mg, yield 73%) was obtained. ¹H NMR (500 MHz, CDCl₃) δ 8.08 (t, J=5.5 Hz, 1H), 7.64 (dd, J=8.6, 2.4 Hz, 1H), 6.69 (d, J=8.6 Hz, 1H), 6.34 (d, J=15.9 Hz, 1H), 6.10 (dt, J=15.8, 7.0 Hz, 1H), 5.81 (dd, J=17.3, 11.0 Hz, 1H), 4.99-4.75 (m, 5H), 4.59 (s, 1H), 3.93 (s, 3H), 2.95 (d, J=6.9 Hz, 2H), 1.99 (ddd, J=15.9, 11.2, 5.0 Hz, 2H), 1.71 (s, 3H), 1.65-1.56 (m, 3H), 1.50-1.41 (m, 3H), 1.01 (s, 3H). ¹³C NMR (126 MHz, CDCl₃) δ 163.43, 153.06, 150.32, 147.77, 145.08, 135.54, 128.25, 127.52, 126.97, 112.29, 110.93, 110.07, 109.01, 53.61, 52.90, 44.41, 40.02 (d, J=11.8 Hz), 38.83, 33.37, 29.84, 27.32, 24.93, 16.76. HRMS (ESI) m/z ([M+H]⁺) calcd for C₂₃H₃₂NO: 338.2478. Found: 338.2475.

### Example 3: Preparation of Compound 3

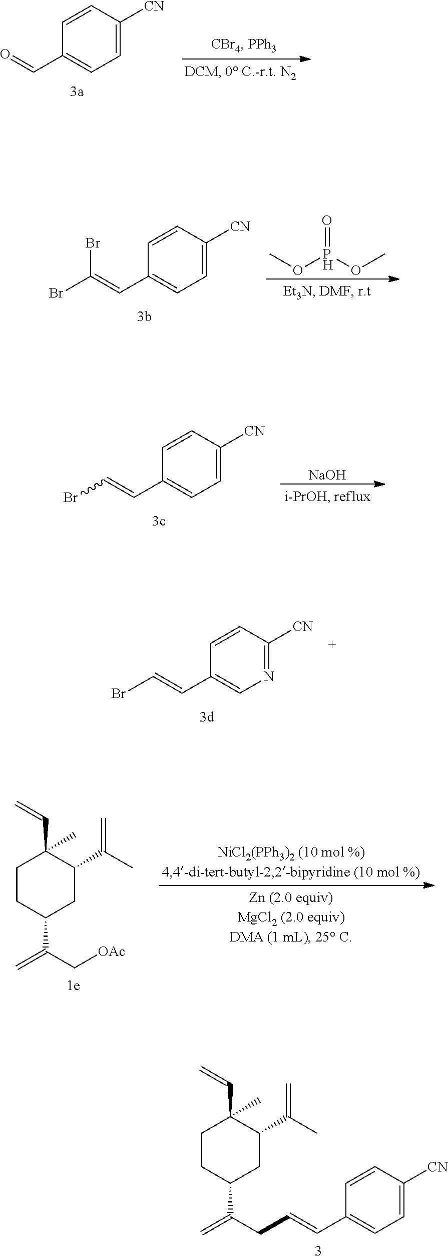

### Compound 3

Referring to the synthesis steps of the Compound 1 in Example 1, a colorless oily liquid 3 (39.7 mg, yield 80%) was obtained. ¹H NMR (500 MHz, CDCl₃) δ 7.75 (d, J=8.3 Hz, 2H), 7.43 (d, J=8.3 Hz, 2H), 6.36 (ddd, J=30.6, 15.8, 11.4 Hz, 2H), 5.81 (dd, J=17.3, 11.0 Hz, 1H), 4.94-4.87 (m, 3H), 4.82 (d, J=5.9 Hz, 2H), 4.59 (s, 1H), 2.99 (d, J=6.8 Hz, 2H), 2.01 (dt, J=18.0, 10.4 Hz, 2H), 1.71 (s, 3H), 1.64-1.56 (m, 3H), 1.52-1.42 (m, 3H), 1.01 (s, 3H). ¹³C NMR (126 MHz, CDCl₃) δ 169.12, 152.70, 150.29, 147.77, 141.53, 131.61, 130.50, 127.86, 126.29, 112.31, 110.10, 109.32, 100.12, 52.89, 44.48, 40.01 (d, J=9.9 Hz), 38.82, 33.36, 29.84, 27.30, 24.93, 16.77. HRMS (ESI) m/z ([M+H]⁺) calcd for C₂₄H₃₀N: 332.2373. Found: 332.2378.

### Example 4: Preparation of Compound 4

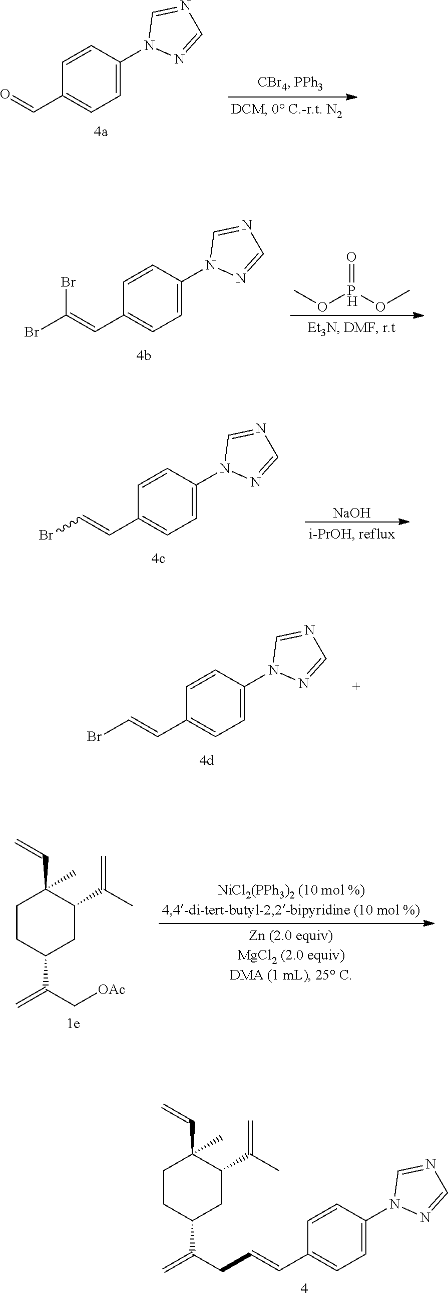

### Compound 4

Referring to the synthesis steps of the Compound 1 in Example 1, a colorless oily liquid 4 (43.1 mg, yield 77%) was obtained. ¹H NMR (500 MHz, CDCl₃) δ 8.54 (s, 1H), 8.10 (s, 1H), 7.61 (d, J=8.5 Hz, 2H), 7.49 (d, J=8.6 Hz, 2H), 6.44 (d, J=15.8 Hz, 1H), 6.29 (dt, J=15.8, 7.0 Hz, 1H), 5.82 (dd, J=17.3, 11.0 Hz, 1H), 4.97-4.87 (m, 3H), 4.83 (d, J=1.4 Hz, 2H), 4.60 (s, 1H), 2.99 (d, J=6.9 Hz, 2H), 2.06-1.97 (m, 2H), 1.71 (s, 3H), 1.64-1.58 (m, 3H), 1.52-1.42 (m, 3H), 1.01 (s, 3H). ¹³C NMR (126 MHz, CDCl₃) 152.81, 152.68, 150.29, 147.77, 140.84, 137.95, 135.73, 130.55, 130.06, 127.38, 120.29, 112.32, 110.11, 109.26, 52.90, 44.49, 40.06, 39.98, 38.78, 33.37, 27.32, 24.94, 16.77. HRMS (ESI) m/z ([M+H]⁺) calcd for C₂₅H₃₂N₃: 374.2591. Found: 374.2590.

### Example 5: Preparation of Compound 5

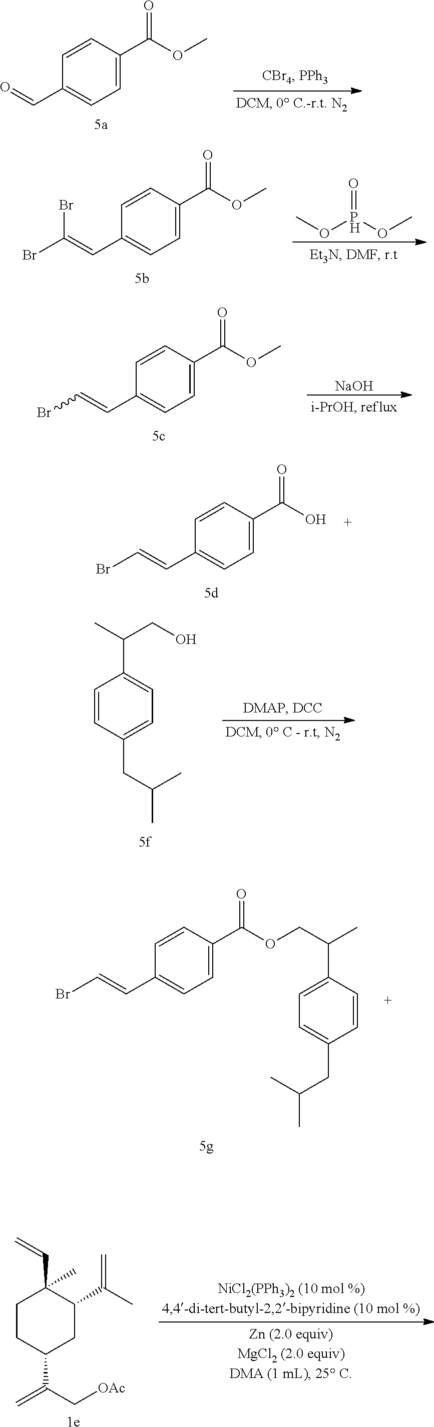

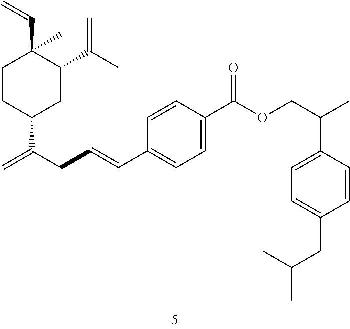

### Intermediate 5g

Referring to the synthesis steps of intermediate 1d in Example 1, a white solid 5d was obtained. In an ice bath, DMAP (37 mg, 0.3 mmol) and DCC (464 mg, 2.25 mmol) were sequentially added to an anhydrous DCM (10 mL) solution of the Compound 5d (358 mg, 1.58 mmol) and Compound 5f (288.4 mg, 1.5 mmol), and stirred overnight. DCM was distilled off under reduced pressure, and an aqueous solution (40 mL) was added to a resulting residue, followed by extraction with ethyl acetate (3×20 mL). A resulting combined organic phase was washed with saturated brine (2×20 mL), and then dried over anhydrous sodium sulfate. The above desiccant was removed by filtration, an obtained filtrate was concentrated under reduced pressure, and an obtained crude product was purified by silica gel column chromatography (eluted with an ethyl acetate/petroleum ether (0.5%) system) to obtain a white solid 5g (581 mg, yield 97%). ¹H NMR (500 MHz, CDCl₃) δ 7.93 (d, J=8.4 Hz, 2H), 7.32 (d, J=8.3 Hz, 2H), 7.18 (d, J=8.0 Hz, 2H), 7.15-7.07 (m, 3H), 6.90 (d, J=14.0 Hz, 1H), 4.42-4.33 (m, 2H), 3.26-3.18 (m, 1H), 2.45 (d, J=7.2 Hz, 2H), 1.84 (dt, J=13.5, 6.8 Hz, 1H), 1.38 (d, J=7.0 Hz, 3H), 0.89 (d, J=6.6 Hz, 6H).

### Compound 5

Referring to the synthesis steps of the Compound 1 in Example 1, a colorless oily liquid 5 (67.6 mg, yield 86%) was obtained. ¹H NMR (500 MHz, CDCl₃) δ 7.91 (d, J=7.9 Hz, 2H), 7.38 (d, J=8.0 Hz, 2H), 7.19 (d, J=7.7 Hz, 2H), 7.10 (d, J=7.7 Hz, 2H), 6.49-6.30 (m, 2H), 5.82 (d, J=6.4 Hz, 1H), 5.01 (s, 1H), 4.93-4.92 (m, 1H), 4.89 (s, 1H), 4.83 (s, 2H), 4.58-4.57 (m, 1H), 4.42-4.31 (m, 2H), 3.21 (dd, J=13.9, 7.0 Hz, 1H), 2.99 (d, J=6.7 Hz, 2H), 2.45 (d, J=7.1 Hz, 2H), 2.09 (s, 3H), 2.01 (d, J=6.8 Hz, 2H), 1.85 (dt, J=13.3, 6.7 Hz, 1H), 1.60 (s, 2H), 1.47 (s, 4H), 1.38 (d, J=6.8 Hz, 3H), 0.99 (s, 3H), 0.90 (d, J=6.6 Hz, 6H). ¹³C NMR (126 MHz, CDCl₃) δ 194.56, 170.79, 166.38, 152.57, 150.20, 150.07, 148.49, 147.40, 142.14, 140.45, 140.09, 131.70, 130.66, 129.96, 129.30, 127.11, 125.95, 112.38, 111.02, 110.13, 109.28, 70.02, 66.20, 52.77, 45.13, 44.42, 41.93, 39.90, 38.79, 33.13, 30.29, 27.13, 24.90, 22.48 (d, J=3.1 Hz), 21.08, 18.13, 16.69. HRMS (ESI) m/z ([M+H]⁺) calcd for C₃₇H₄₉O₂: 525.3727. Found: 525.3722.

### Example 6: Preparation of Compound 6

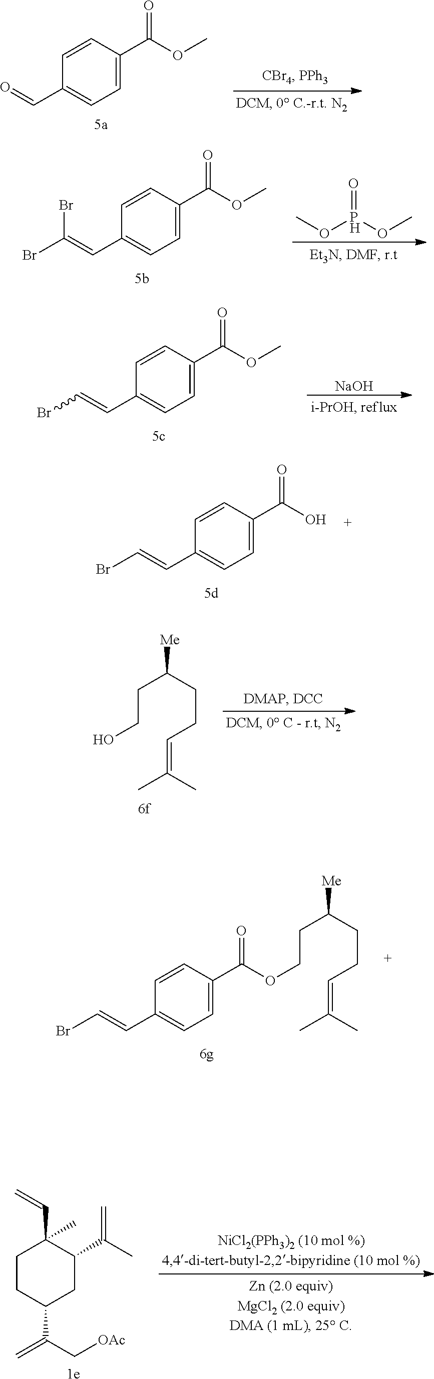

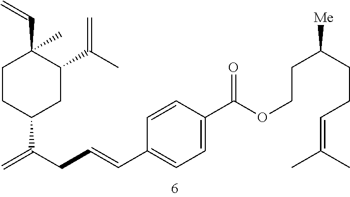

### Intermediate 6g

Referring to the synthesis steps of the intermediate 5g in Example 5, a colorless oily liquid 6g (267 mg, yield 82%) was obtained. ¹H NMR (500 MHz, CDCl₃) δ 7.98 (d, J=8.3 Hz, 2H), 7.34 (d, J=8.3 Hz, 2H), 7.13 (d, J=14.0 Hz, 1H), 6.91 (d, J=14.0 Hz, 1H), 5.09 (t, J=7.1 Hz, 1H), 4.40-4.29 (m, 2H), 1.99 (dd, J=22.5, 7.6 Hz, 2H), 1.80 (dt, J=12.5, 6.3 Hz, 1H), 1.66 (s, 3H), 1.59 (s, 3H), 1.34-1.28 (m, 2H), 1.24 (dd, J=15.0, 7.7 Hz, 2H), 0.96 (d, J=6.6 Hz, 3H).

### Compound 6

Referring to the synthesis steps of the Compound 1 in Example 1, a colorless oily liquid 6 (63.7 mg, yield 87%) was obtained. ¹H NMR (500 MHz, CDCl₃) δ 7.97 (d, J=7.9 Hz, 2H), 7.41 (d, J=8.0 Hz, 2H), 6.50-6.29 (m, 2H), 5.82 (dd, J=17.4, 10.8 Hz, 1H), 5.10 (dd, J=6.9, 5.9 Hz, 1H), 4.92 (s, 1H), 4.91 (s, 1H), 4.89 (d, J=2.6 Hz, 1H), 4.82 (d, J=5.1 Hz, 2H), 4.60 (s, 1H), 4.46-4.27 (m, 2H), 2.99 (d, J=6.8 Hz, 2H), 2.07-1.97 (m, 4H), 1.92 (d, J=10.1 Hz, 1H), 1.86-1.79 (m, 1H), 1.75 (d, J=9.2 Hz, 1H), 1.71 (s, 3H), 1.68 (s, 3H), 1.61 (s, 3H), 1.56 (d, J=8.1 Hz, 1H), 1.51-1.44 (m, 3H), 1.31 (dd, J=17.7, 7.5 Hz, 4H), 1.01 (s, 3H), 0.97 (d, J=6.5 Hz, 3H). ¹³C NMR (126 MHz, CDCl₃) δ 166.64, 152.66, 150.27, 147.72, 142.14, 131.72, 131.48, 130.72, 130.00, 129.01 125.99, 124.71, 112.30, 110.08, 109.31, 63.53, 52.87, 44.47, 40.00 (d, J=10.8 Hz), 38.84, 37.13, 35.66, 35.06, 33.35, 29.72, 27.30, 25.84, 25.54, 24.93, 19.65, 17.80, 16.75. HRMS (ESI) m/z ([M+H]⁺) calcd for C₃₄H₄₉O₂: 489.3727. Found: 489.3730.

### Example 7: Preparation of Compound 7

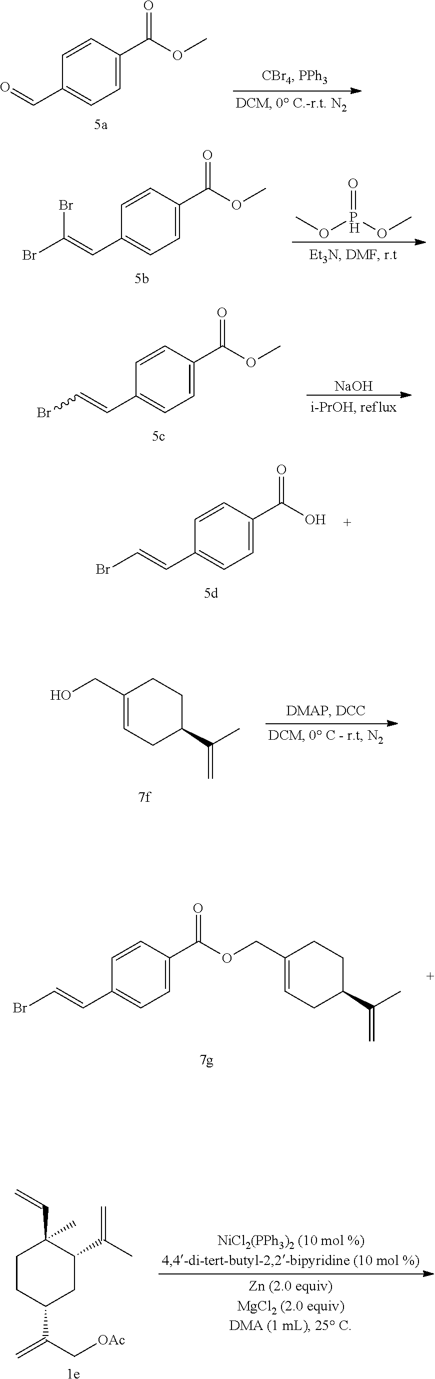

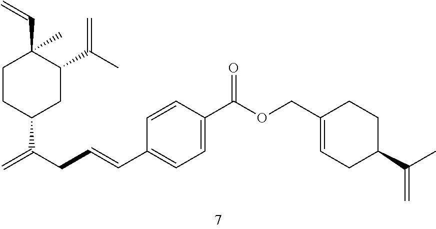

### Intermediate 7g

Referring to the synthesis steps of the intermediate 5g in Example 5, a colorless oily liquid 7g (281 mg, yield 89%) was obtained. ¹H NMR (500 MHz, CDCl₃) δ 8.00 (d, J=8.2 Hz, 2H), 7.35 (d, J=8.3 Hz, 2H), 7.13 (d, J=14.0 Hz, 1H), 6.91 (d, J=14.0 Hz, 1H), 5.84 (s, 1H), 4.73-4.70 (m, 3H), 2.17 (d, J=3.6 Hz, 1H), 1.93-1.89 (m, 2H), 1.74 (s, 3H), 1.58-1.49 (m, 2H), 1.30 (d, J=9.8 Hz, 2H).

### Compound 7

Referring to the synthesis steps of the Compound 1 in Example 1, a colorless oily liquid 7 (59.5 mg, yield 82%) was obtained. ¹H NMR (500 MHz, CDCl₃) δ 7.97 (d, J=8.3 Hz, 2H), 7.40 (d, J=8.3 Hz, 2H), 6.38 (dt, J=15.8, 12.4 Hz, 2H), 5.82-5.80 (m, 2H), 5.00 (s, 1H), 4.91 (s, 1H), 4.88 (d, J=2.1 Hz, 1H), 4.81 (s, 2H), 4.71 (dd, J=8.9, 5.0 Hz, 4H), 4.57 (s, 1H), 2.98 (d, J=6.7 Hz, 2H), 2.17 (d, J=5.1 Hz, 3H), 2.08 (s, 3H), 2.01 (s, 2H), 1.90 (d, J=10.0 Hz, 3H), 1.70-1.69 (m, 3H), 1.59 (s, 2H), 1.46 (d, J=3.4 Hz, 4H), 1.29 (s, 1H), 1.00-0.99 (m, 3H). 13C NMR (126 MHz, CDCl₃) δ 194.56, 170.79, 154.90, 152.57, 150.06, 148.48, 147.40, 132.89, 131.73, 130.66, 130.02, 125.96, 125.59, 112.36, 111.01, 110.12, 108.90, 68.78, 66.20, 55.80, 52.76, 48.46, 45.71, 44.42, 41.92, 40.95, 39.89, 38.79, 36.51, 35.00, 33.12, 30.56. HRMS (ESI) m/z ([M+H]⁺) calcd for C₃₄H₄₅O₂: 485.3414. Found: 485.3409.

### Example 8: Preparation of Compound 8

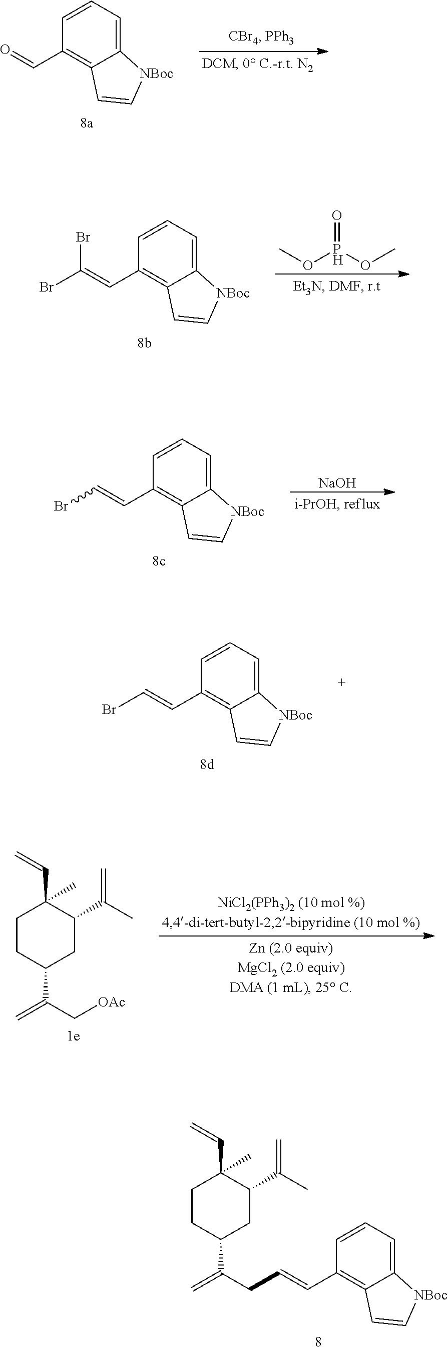

### Compound 8

Referring to the synthesis steps of the Compound 1 in Example 1, a colorless oily liquid 8 (50.1 mg, yield 75%) was obtained. ¹H NMR (500 MHz, CDCl₃) δ 8.01 (d, J=7.7 Hz, 1H), 7.59 (d, J=3.6 Hz, 1H), 7.32 (d, J=7.5 Hz, 1H), 7.24 (t, J=3.9 Hz, 1H), 6.72 (d, J=3.3 Hz, 2H), 6.32 (dt, J=15.7, 7.1 Hz, 1H), 5.80 (dd, J=17.4, 10.9 Hz, 1H), 4.93-4.77 (m, 5H), 4.58 (s, 1H), 3.02 (d, J=7.0 Hz, 2H), 2.00 (dd, J=12.0, 3.9 Hz, 2H), 1.70 (s, 3H), 1.65 (s, 12H), 1.46 (ddd, J=9.0, 7.8, 2.6 Hz, 3H), 1.00 (s, 3H). ¹³C NMR (126 MHz, CDCl₃) δ 153.17, 150.37, 147.82, 135.65, 134.23, 130.42 (d, J=8.5 Hz), 128.63, 125.92, 124.48, 119.37, 115.42, 114.66, 113.93, 112.29, 110.04, 109.06, 105.62, 83.83, 52.92, 44.45, 40.05 (d, J=12.6 Hz), 39.20, 33.41, 29.85, 28.34, 27.36, 24.94, 16.78. HRMS (ESI) m/z ([M−H]⁻) calcd for C₃₀H₃₈NO₂: 444.2908. Found: 444.2908.

### Example 9: Preparation of Compound 9

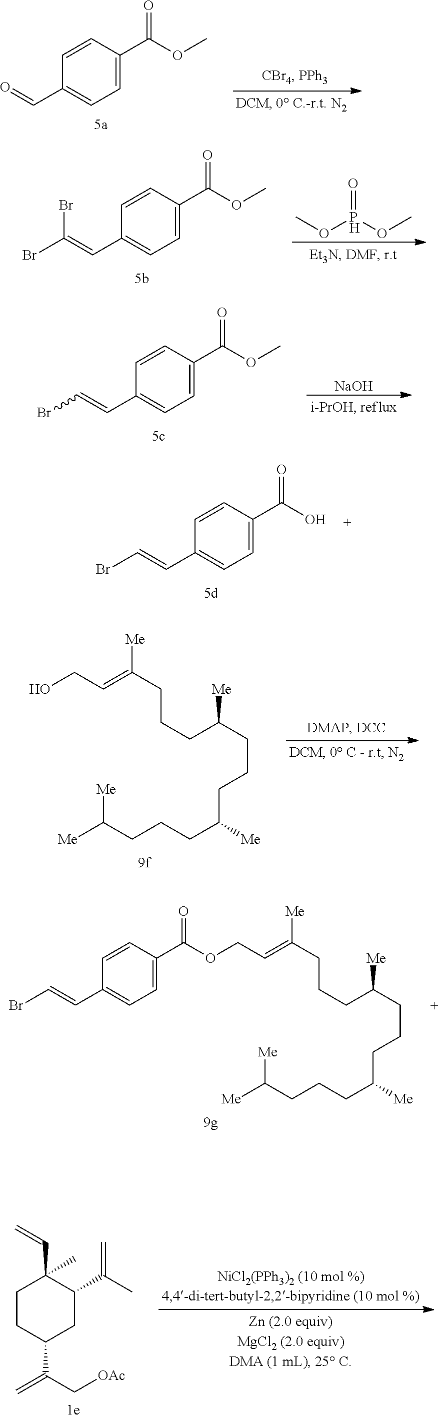

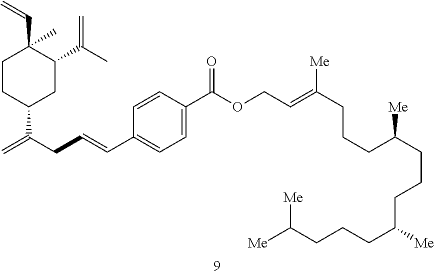

### Intermediate 9g

Referring to the synthesis steps of the intermediate 5g in Example 5, a colorless oily liquid 9g (416 mg, yield 69%) was obtained. ¹H NMR (500 MHz, CDCl₃) δ 7.99 (d, J=8.4 Hz, 2H), 7.34 (d, J=8.3 Hz, 2H), 7.13 (d, J=14.1 Hz, 1H), 6.90 (d, J=14.0 Hz, 1H), 5.45 (t, J=7.0 Hz, 1H), 4.83 (d, J=7.0 Hz, 2H), 2.04 (d, J=5.1 Hz, 2H), 1.75 (s, 3H), 1.51 (dd, J=13.3, 6.7 Hz, 1H), 1.39-1.11 (m, 20H), 0.88-0.81 (m, 12H).

### Compound 9

Referring to the synthesis steps of the Compound 1 in Example 1, a colorless oily liquid 9 (73.6 mg, yield 78%) was obtained. ¹H NMR (500 MHz, CDCl₃) δ 7.98 (d, J=8.4 Hz, 2H), 7.40 (d, J=8.4 Hz, 2H), 6.54-6.26 (m, 2H), 5.82 (dd, J=17.3, 11.0 Hz, 1H), 5.46 (dd, J=7.0, 6.0 Hz, 1H), 4.93-4.89 (m, 2H), 4.83 (dd, J=5.9, 4.0 Hz, 3H), 4.60 (s, 1H), 3.20 (dt, J=13.3, 5.0 Hz, 1H), 2.99 (d, J=6.8 Hz, 2H), 2.07-1.98 (m, 5H), 1.95-1.89 (m, 2H), 1.75 (s, 3H), 1.71 (s, 3H), 1.63-1.57 (m, 5H), 1.48 (ddd, J=13.1, 9.9, 5.1 Hz, 8H), 1.16-1.12 (m, 5H), 1.09-1.05 (m, 5H), 1.01 (s, 3H), 0.86 (d, J=5.5 Hz, 12H). ¹³C NMR (126 MHz, CDCl₃) δ 166.66, 152.68, 150.29, 147.74, 142.87, 142.13, 131.69, 130.74, 130.07, 129.03, 125.98, 118.37, 112.31, 110.08, 109.32, 61.98, 55.89, 52.88, 44.47, 40.05, 39.79, 39.52, 38.85, 37.57, 37.51, 37.44, 36.77, 35.07, 33.36, 32.87 (d, J=15.3 Hz), 29.85, 28.12, 27.30, 25.60, 25.19, 24.94, 24.61, 22.82 (d, J=11.7 Hz), 19.88 (d, J=3.7 Hz), 16.69 (d, J=18.4 Hz), 14.27. HRMS (ESI) m/z ([M+H]⁺) calcd for C₄₄H₆₉O₂: 629.5292. Found: 629.5291.

### Example 10: Preparation of Compound 10

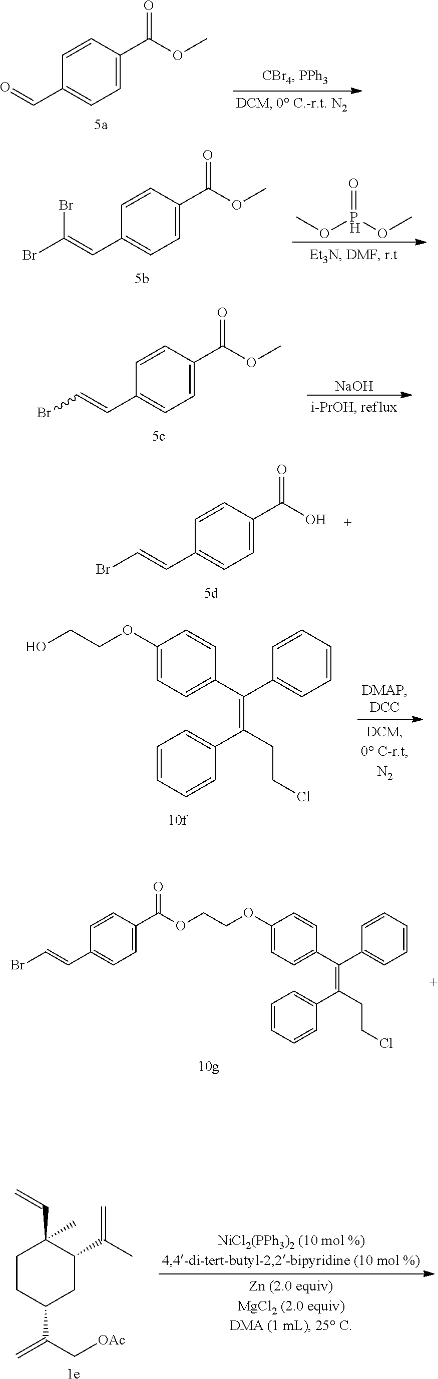

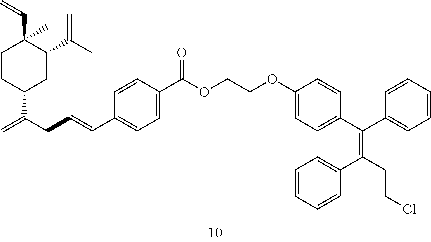

### Intermediate 10g

Referring to the synthesis steps of the intermediate 5g in Example 5, a white solid log (1.07 g, yield 91%) was obtained. ¹H NMR (500 MHz, CDCl₃) δ 7.97 (dd, J=8.5, 1.6 Hz, 2H), 7.39-7.34 (m, 2H), 7.34-7.30 (m, 2H), 7.30-7.27 (m, 2H), 7.20 (dd, J=8.1, 6.8 Hz, 2H), 7.16-7.10 (m, 4H), 6.91 (d, J=14.1 Hz, 1H), 6.82-6.76 (m, 2H), 6.62-6.56 (m, 2H), 4.57 (dd, J=5.5, 3.9 Hz, 2H), 4.16 (dd, J=5.6, 3.9 Hz, 2H), 3.41 (t, J=7.4 Hz, 2H), 2.92 (t, J=7.5 Hz, 2H).

### Compound 10

Referring to the synthesis steps of the Compound 1 in Example 1, a colorless oily liquid 10 (86.4 mg, yield 81%) was obtained. ¹H NMR (500 MHz, CDCl₃) δ 7.96 (d, J=8.2 Hz, 2H), 7.42-7.36 (m, 4H), 7.31 (d, J=6.2 Hz, 3H), 7.23-7.19 (m, 2H), 7.16 (d, J=7.2 Hz, 3H), 6.82 (d, J=8.7 Hz, 2H), 6.61 (d, J=8.7 Hz, 2H), 6.51-6.31 (m, 2H), 5.86-5.80 (m, 1H), 5.11 (d, J=13.8 Hz, 1H), 4.92 (s, 1H), 4.91 (s, 1H), 4.84 (d, J=6.9 Hz, 2H), 4.61 (s, 1H), 4.59-4.56 (m, 2H), 4.20-4.14 (m, 2H), 3.43 (t, J=7.4 Hz, 2H), 3.00 (d, J=8.7 Hz, 2H), 2.95-2.91 (m, 2H), 2.05-2.00 (m, 2H), 1.73 (s, 3H), 1.62 (dd, J=7.5, 3.3 Hz, 2H), 1.51-1.45 (m, 4H), 1.03 (s, 3H). ¹³C NMR (126 MHz, CDCl₃) δ 166.44, 156.86, 152.58, 150.24, 148.88, 147.68, 142.94, 142.41, 141.75, 141.03, 135.41 (d, J=5.3 Hz), 131.93, 131.86, 130.62, 130.16, 129.63, 129.49, 128.46, 128.34, 127.07, 126.73, 125.98, 113.72, 113.13, 112.29, 110.07, 109.33, 79.52, 65.88, 63.33, 52.83, 44.43, 42.93, 41.65, 39.96 (d, J=9.6 Hz), 38.75 (d, J=14.8 Hz), 33.31, 29.81, 27.26, 24.92. HRMS (ESI) m/z ([M+Na]⁺) calcd for C₄₈H₅₁ClNaO₃: 733.3419. Found: 733.3482.

### Example 11: Experiment of Tumor Cell Proliferation Inhibition

### Evaluation of Antitumor Activity In Vitro

### 1. Experimental Equipment and Reagents

### 1.1 Instrument

Biological safety cabinet (Shanghai Bio-Gene Biotechnology Co., Ltd.), constant-temperature carbon dioxide incubator (THERMO), enzyme-linked immunoassay analyzer (Spark), inverted microscope (Nikon), a set of pipette guns (eppendorf), and centrifuge (beckman coulter).

### 1.2 Reagents

McCoy'S 5A (Zhejiang Cienry Biotechnology Co., Ltd.), RPMI 1640 (Zhejiang Cienry Biotechnology Co., Ltd.), Fatal Bovine Serum (BI), PBS (Zhejiang Cienry Biotechnology Co., Ltd.), Trypsin (Zhejiang Cienry Biotechnology Co., Ltd.), DMSO (Coolaber), and CCK-8 (Coolaber).

### 1.3 Cell Lines

Human colon cancer cells (HCT116), human lung cancer cells (A549).

### 2. Experimental Methods

1) The test cells in a logarithmic growth phase were trypsinized and counted, inoculated in a 96-well culture plate at a concentration of 5×10⁴/mL, 100 μL per well (5×10³ cells per well), and incubated at 37° C. in a 5% CO₂ incubator for 24 h.

2) A drug to be tested was diluted to different concentrations with McCoy'S 5A or RPMI 1640 complete medium. The experimental group was replaced with a culture solution containing different concentrations of the tested samples, and the control group was replaced with a culture solution containing the same volume of solvent (DMSO). Three parallel wells were set up in each group, and the culture was continued for 48 h in a 5% CO₂ incubator at 37° C.

HCT116 cells used the McCoy'S 5A complete medium, and A549 cells used the RPMI 1640 complete medium.

3) 10 μL of a CCK-8 solution was added to each well, incubated at 37° C. for 1 h to 4 h, and an absorbance (OD value) of each well at 490 nm was measured with a microplate reader.

4) A survival rate and an inhibition rate were calculated with the following formulas:

Cell survival rate=[(Aₛ−A_{b})/(A_{c}−A_{b})]×100%

Inhibition rate=[(A_{c}−Aₛ)/(A_{c}−A_{b})]×100%

An S-type dose-survival curve was plotted using GraphPad Prism 7.0 software and a nonlinear regression model, and IC₅₀ values were calculated.
Aₛ: absorbance of the experimental well (medium containing cells, CCK-8, and the drug to be tested)
A_{c}: absorbance of control wells (medium containing cells, CCK-8, and solvent (DMSO))
A_{b}: absorbance of blank wells (medium without cells and drug to be tested, CCK-8)

### 3. Experimental Results

The inhibitory effects of the target compound and the positive control drug β-elemene on the proliferation of two tumor cells were determined according to the above experimental method. The results were shown in Table 1.

TABLE 1
Effect of target compounds on tumor cell inhibition rate

| | Anti-proliferative activity at 100 μM | |
|---|---|---|
| | by concentration (%) | |
| Compound No. | HCT116 | A549 |
| 4 | 99.8 ± 0.08 | 97.5 ± 0.4 |
| 6 | 21.7 ± 0.8 | 14.4 ± 0.5 |
| 10 | 13.5 ± 0.3 | 40.4 ± 0.6 |
| β-elemene | 0.6 ± 0.5 | 10.6 ± 0.5 |

100 μM of the compound acted on tumor cells for 48 h.

The results showed that Compound 4 showed an anti-tumor cell proliferation effect significantly stronger than that of the positive control β-elemene.

The compound 4 was selected to determine the IC₅₀ value according to the above experimental method. The results were shown in Table 2.

TABLE 2
IC₅₀ of target compounds on tumor cells

| | IC₅₀ (μM) | |
|---|---|---|
| Compound No. | HCT116 | A549 |
| 4 | 50.9 ± 0.8 | 57.21 ± 2.95 |
| β-elemene | 781.9 ± 51 | 227.0 ± 1.2 |

The results showed that Compound 4 exhibited significantly stronger cell proliferation-inhibiting activity than the positive control β-elemene.

In addition, it should be understood that various changes or modifications may be made to the present disclosure by those skilled in the art after reading the above teaching content of the present disclosure, and these equivalent forms also fall within the scope defined by the appended claims of the present disclosure.

What is claimed is:

1. A vinylation coupled derivative of β-elemene, or an optical isomer, a racemate, a single enantiomer, and a diastereomer thereof, or a pharmaceutically acceptable salt, a prodrug, a deuterated derivative, a hydrate, and a solvate thereof, wherein the vinylation coupled derivative of β-elemene has a structure shown in formula (I): 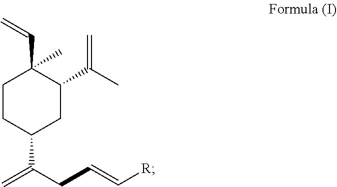
R is selected from the group consisting of aryl, heteroaryl, and alkyl in formula (I).

2. The vinylation coupled derivative of β-elemene, or an optical isomer, a racemate, a single enantiomer, and a diastereomer thereof, or a pharmaceutically acceptable salt, a prodrug, a deuterated derivative, a hydrate, and a solvate thereof according to claim 1, wherein R in the structure shown in Formula (I) of the vinylation coupled derivative of β-elemene is any one independently selected from the group consisting of the following structural fragments:

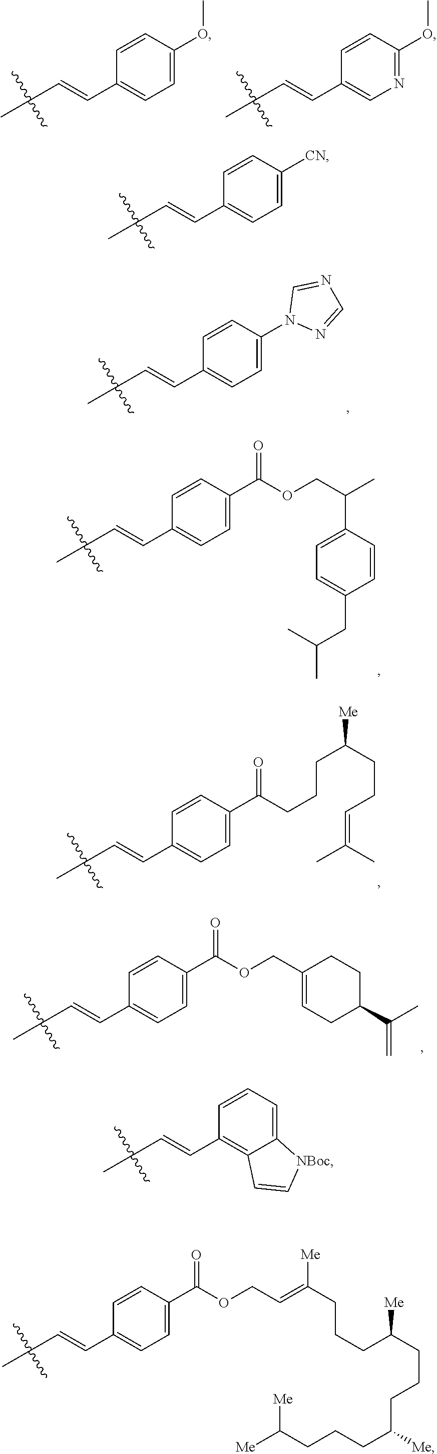

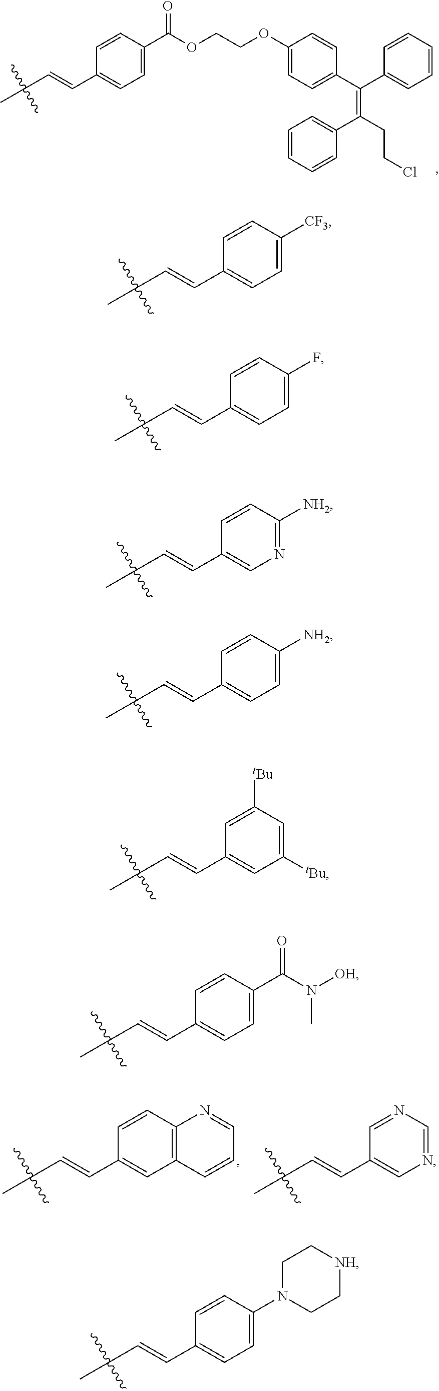

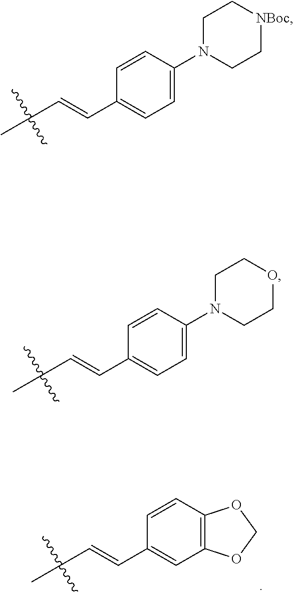 [SMILES: */C=C/c1ccc(N2CCN(C(=O)OC(C)(C)C)CC2)cc1 ; */C=C/c1ccc(N2CCOCC2)cc1 ; */C=C/c1ccc2c(c1)OCO2]

3. The vinylation coupled derivative of β-elemene, or an optical isomer, a racemate, a single enantiomer, and a diastereomer thereof, or a pharmaceutically acceptable salt, a prodrug, a deuterated derivative, a hydrate, and a solvate thereof according to claim 1, wherein the vinylation coupled derivative of β-elemene has a structure shown in any one of Compounds 1 to 22:

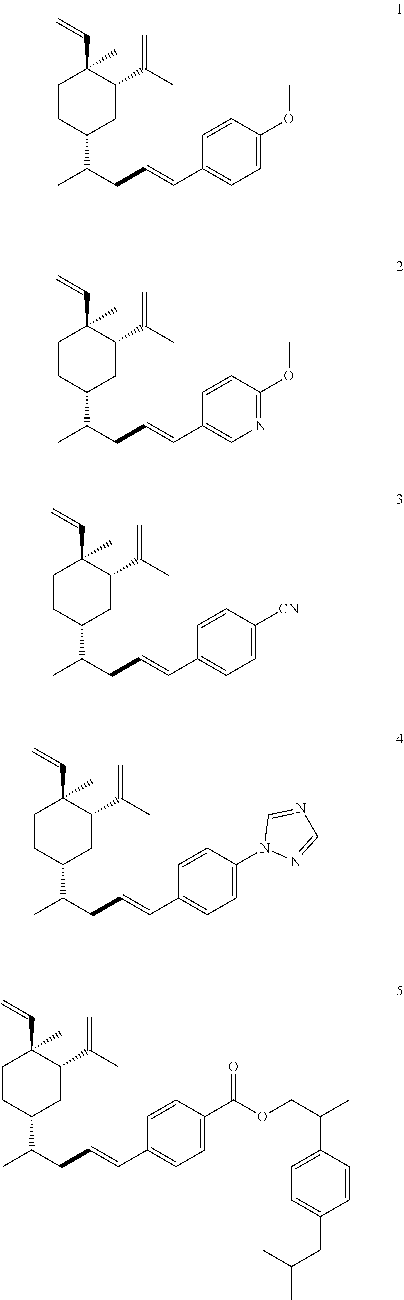

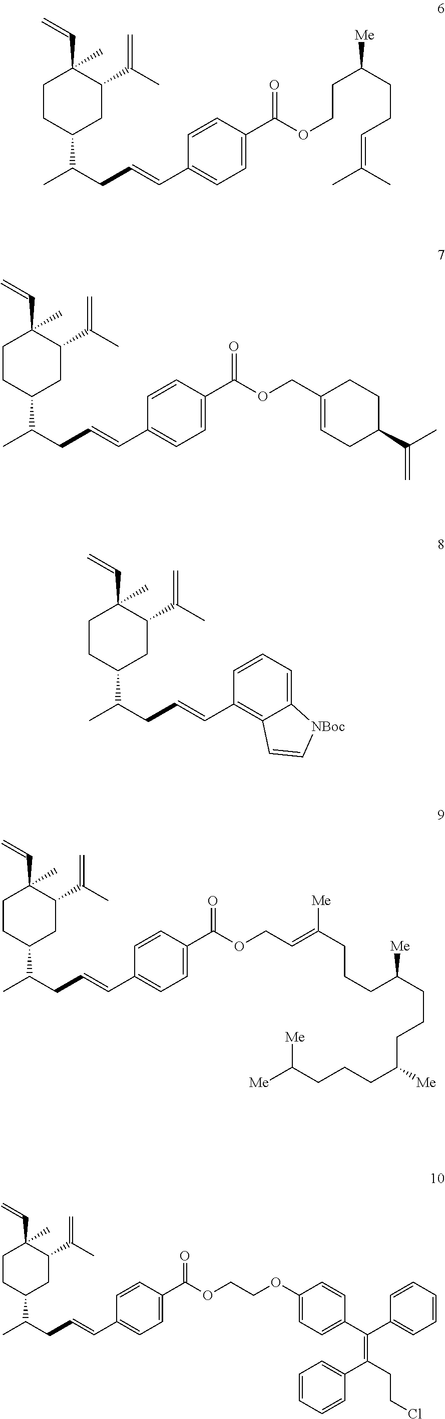

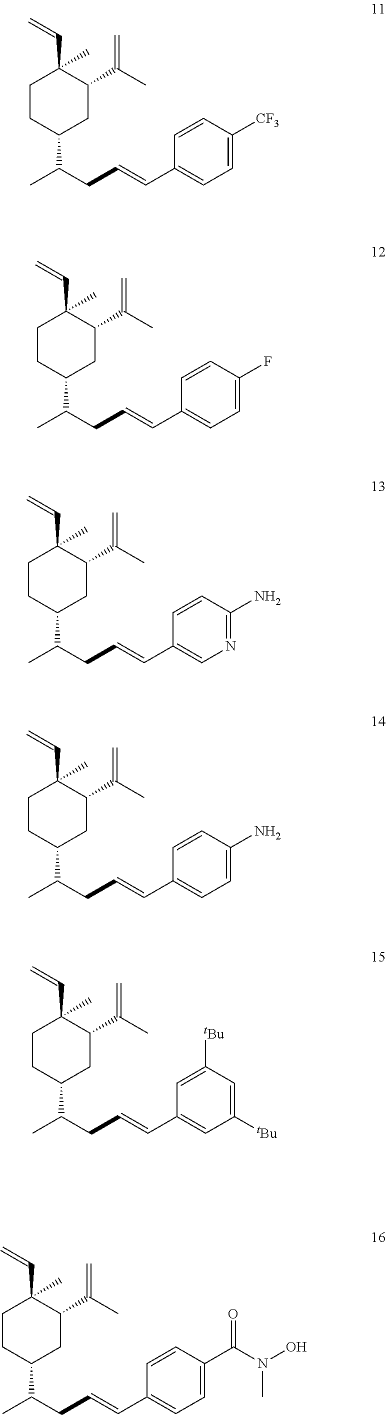

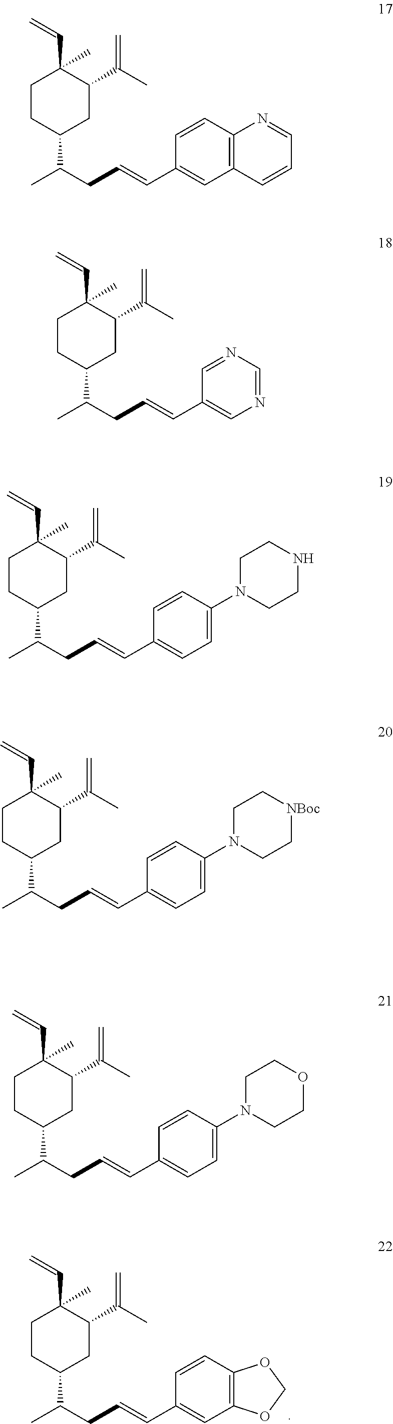

4. A preparation method of the vinylation coupled derivative of β-elemene according to claim 1, wherein the preparation method has the following synthetic route: 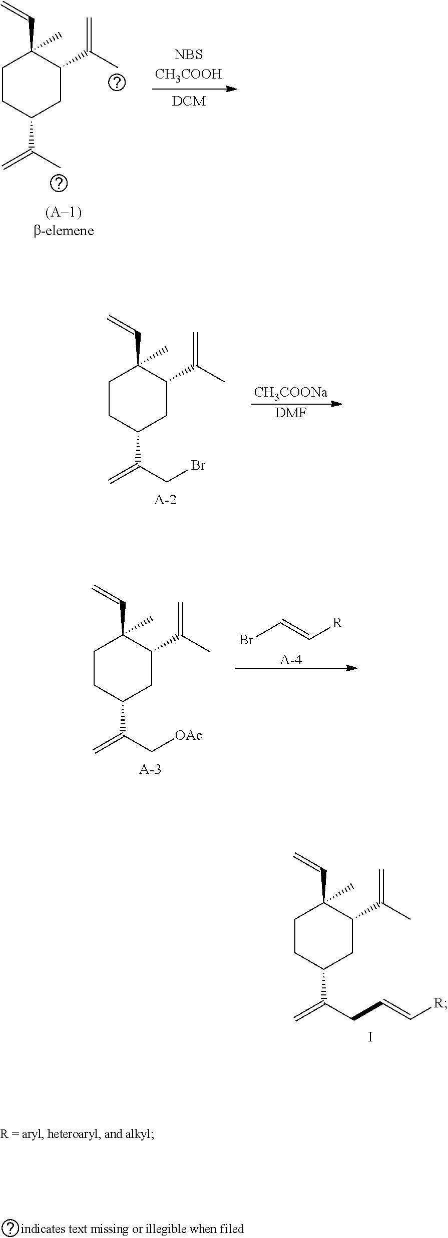
and
the preparation method comprises the following steps:
(1) subjecting β-elemene A-1 to monobromine substitution at an allyl position to obtain an intermediate A-2;
(2) subjecting the intermediate A-2 to selective nucleophilic substitution to obtain an intermediate A-3; and
(3) allowing the intermediate A-3 and alkenyl bromide A-4 to have vinylation coupling to obtain the vinylation coupled derivative of β-elemene having a structure shown in Formula (I); wherein
R is any one independently selected from the group consisting of the following structural fragments:

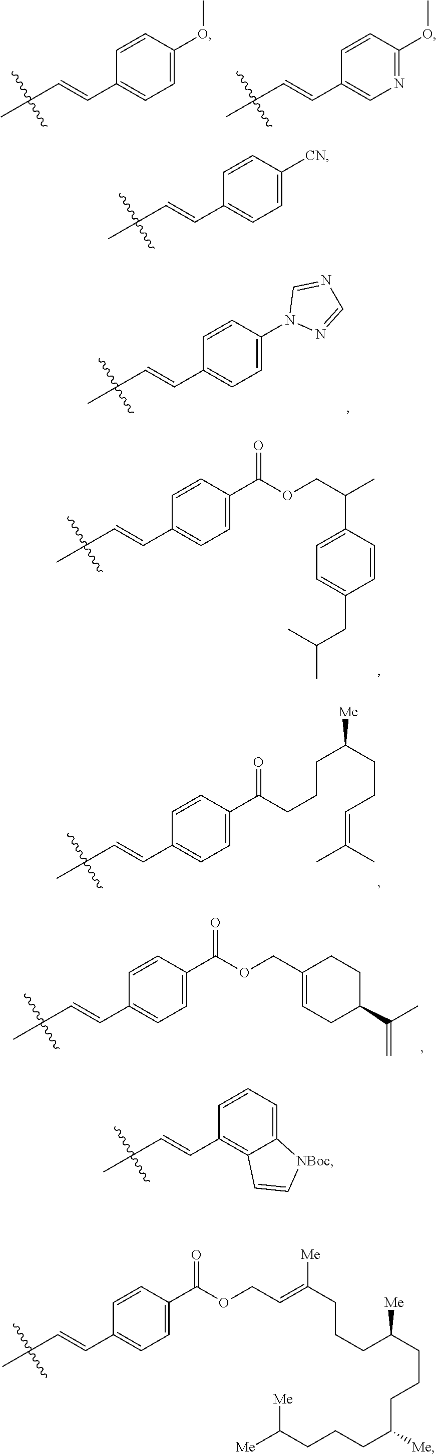

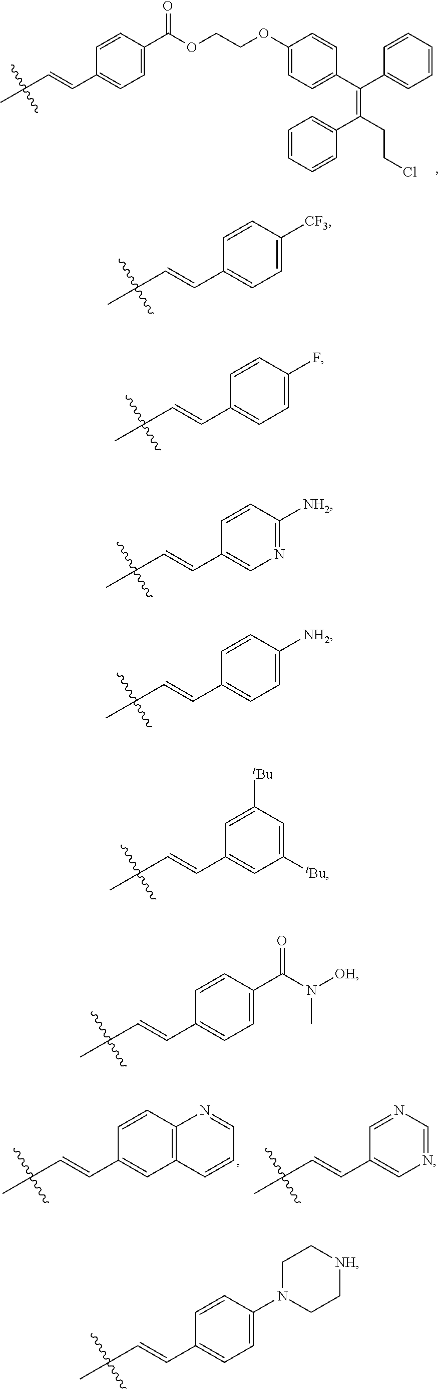

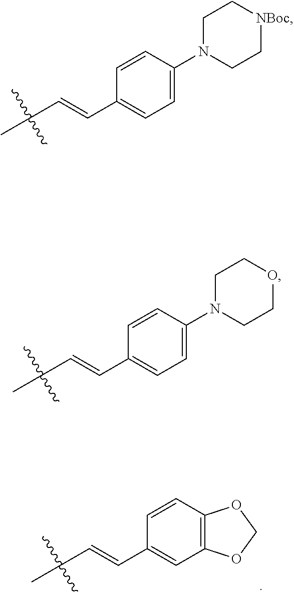 [SMILES: */C=C/c1ccc(N2CCN(C(=O)OC(C)(C)C)CC2)cc1 ; */C=C/c1ccc(N2CCOCC2)cc1 ; */C=C/c1ccc2c(c1)OCO2]

5. A method for treating a tumor, comprising administering the vinylation coupled derivative of β-elemene, or an optical isomer, a racemate, a single enantiomer, and a diastereomer thereof, or a pharmaceutically acceptable salt, a prodrug, a deuterated derivative, a hydrate, and a solvate thereof according to claim 1 to a subject in need.

6. The method according to claim 5, wherein the antitumor drug is used for treating colon cancer, lung cancer, liver cancer, gastric cancer, prostate cancer, ovarian cancer, breast cancer, or glioma.

7. The method according to claim 5, wherein the vinylation coupled derivative of β-elemene, or an optical isomer, a racemate, a single enantiomer, and a possible diastereomer thereof, or a pharmaceutically acceptable salt, a prodrug, a deuterated derivative, a hydrate, and a solvate thereof is applied by oral administration, intratumoral administration, rectal administration, parenteral administration, and topical administration.

8. The method according to claim 7, wherein a solid dosage form for the oral administration comprises a capsule, a tablet, a pill, a powder, and a granule;
in the solid dosage form, the vinylation coupled derivative of β-elemene, or an optical isomer, a racemate, a single enantiomer, and a possible diastereomer thereof, or a pharmaceutically acceptable salt, a prodrug, a deuterated derivative, a hydrate, and a solvate thereof is mixed with at least one inert excipient or carrier; the inert excipient or carrier is selected from the group consisting of sodium citrate and dicalcium phosphate; alternatively, the vinylation coupled derivative of β-elemene, or an optical isomer, a racemate, a single enantiomer, and a possible diastereomer thereof, or a pharmaceutically acceptable salt, a prodrug, a deuterated derivative, a hydrate, and a solvate thereof is mixed with a filler or a solubilizer, a binder, a humectant, a disintegrant, a retarding solvent, an absorption accelerator, a wetting agent, an adsorbent, and a lubricant; the filler or the solubilizer is selected from the group consisting of starch, lactose, sucrose, glucose, mannitol, and silicic acid; the binder is selected from the group consisting of hydroxymethylcellulose, an alginate, gelatin, polyvinylpyrrolidone, sucrose, and arabic gum; the humectant is glycerin; the disintegrant is selected from the group consisting of agar, calcium carbonate, potato starch or tapioca starch, alginic acid, a complex silicate, and sodium carbonate; the retarding solvent is paraffin; the absorption accelerator is a quaternary ammonium compound; the wetting agent is selected from the group consisting of cetyl alcohol and glyceryl monostearate; the adsorbent is kaolin; and the lubricant is selected from the group consisting of talc, calcium stearate, magnesium stearate, solid polyethylene glycol, and sodium lauryl sulfate and a mixture thereof.

9. The method according to claim 5, wherein a daily dosage is 1 mg to 5,000 mg for a person weighing 60 kg.

10. An antitumor drug, comprising a safe and effective dosage of the vinylation coupled derivative of β-elemene, or an optical isomer, a racemate, a single enantiomer, and a possible diastereomer thereof, or a pharmaceutically acceptable salt, a prodrug, a deuterated derivative, a hydrate, and a solvate thereof according to claim 1.

11. The antitumor drug according to claim 10, wherein R in the structure shown in Formula (I) of the vinylation coupled derivative of β-elemene is any one independently selected from the group consisting of the following structural fragments:

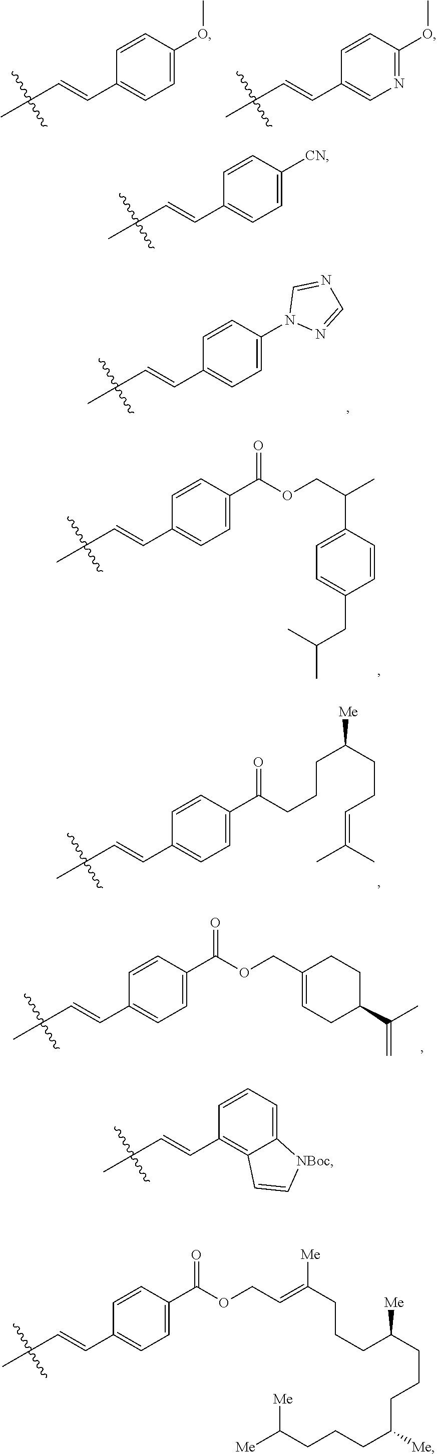

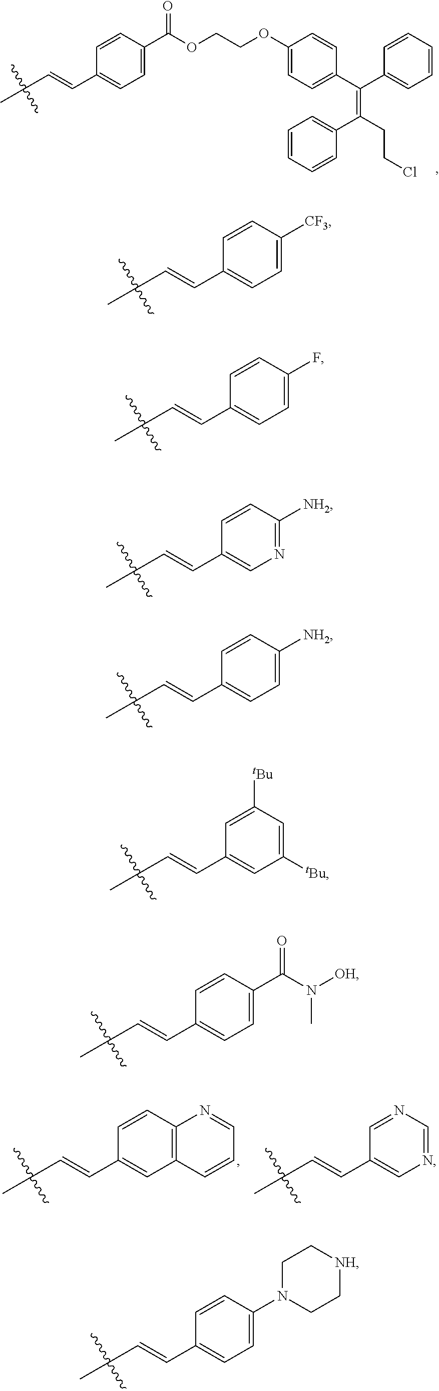

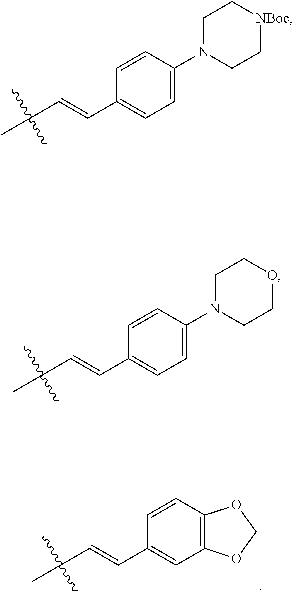 [SMILES: */C=C/c1ccc(N2CCN(C(=O)OC(C)(C)C)CC2)cc1 ; */C=C/c1ccc(N2CCOCC2)cc1 ; */C=C/c1ccc2c(c1)OCO2]

12. The antitumor drug according to claim 10, wherein the vinylation coupled derivative of β-elemene has a structure shown in any one of Compounds 1 to 22:

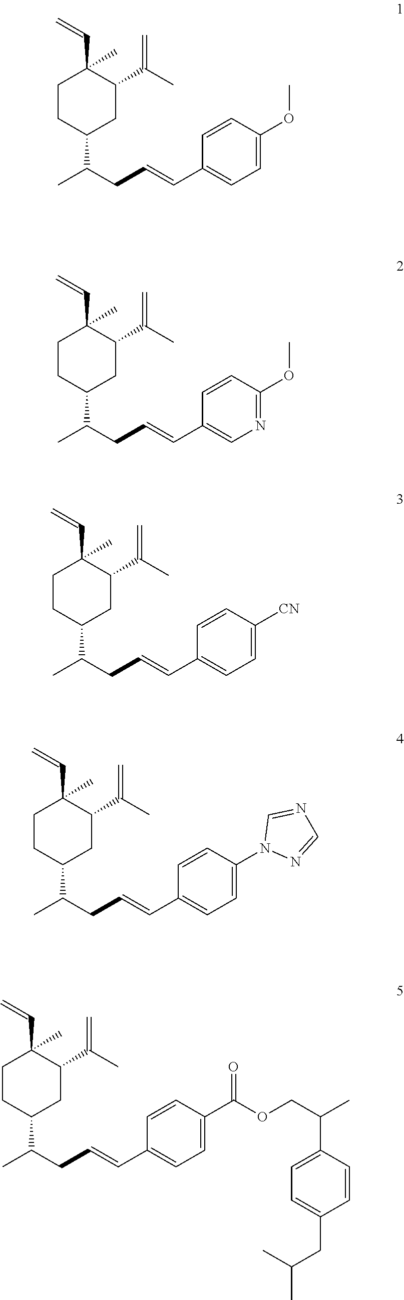

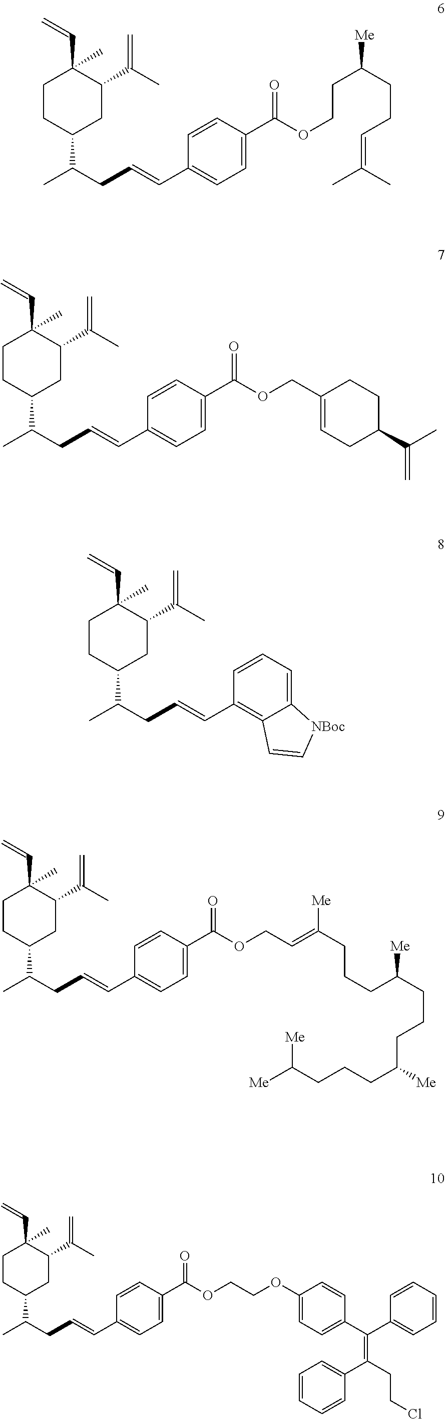

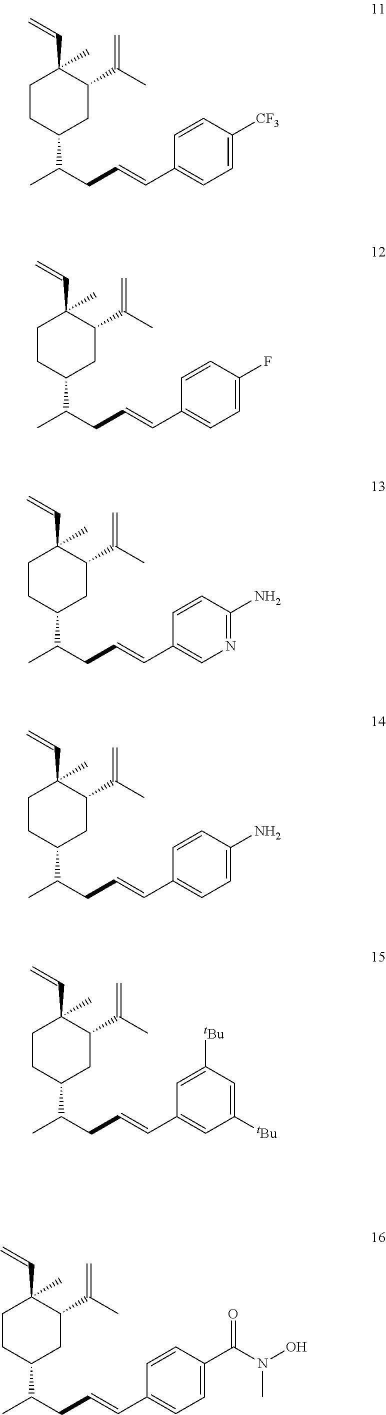

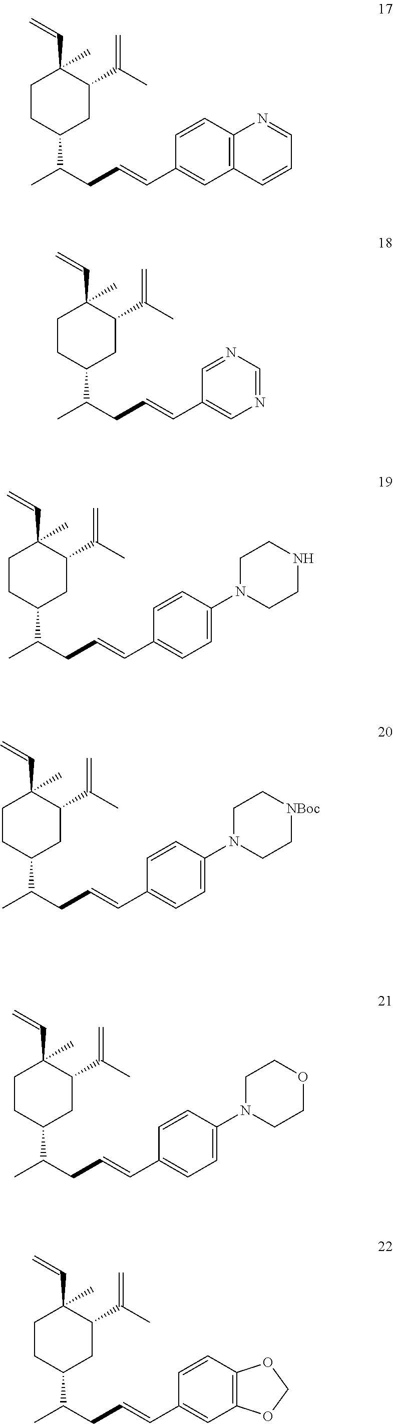

13. The antitumor drug according to claim 10, further comprising a pharmaceutically acceptable excipient or carrier.

14. The antitumor drug according to claim 11, further comprising a pharmaceutically acceptable excipient or carrier.

15. The antitumor drug according to claim 12, further comprising a pharmaceutically acceptable excipient or carrier.

16. The antitumor drug according to claim 10, wherein the antitumor drug is used for treating colon cancer and lung cancer.

17. The antitumor drug according to claim 11, wherein the antitumor drug is used for treating colon cancer and lung cancer.

18. The antitumor drug according to claim 10, wherein the antitumor drug carries the vinylation coupled derivative of β-elemene, or an optical isomer, a racemate, a single enantiomer, and a possible diastereomer thereof, or a pharmaceutically acceptable salt, a prodrug, a deuterated derivative, a hydrate, and a solvate thereof at 1 mg/dose to 2,000 mg/dose; and one dose refers to one capsule or one tablet.

19. The antitumor drug according to claim 10, wherein the pharmaceutically acceptable carrier comprises cellulose and a derivative thereof, gelatin, talc, a solid lubricant, calcium sulfate, vegetable oil, a polyol, an emulsifier, a wetting agent, a coloring agent, a flavoring agent, a stabilizer, an antioxidant, a preservative, and pyrogen-free water.

20. The method according to claim 8, wherein the tablet, the capsule, the pill, and the granule each are prepared from a coating and a shell material.
